(19) United States
(12) Patent Application Publication
(10) Pub. No.: US 2022/0363720 A1
(43) Pub. Date: Nov. 17, 2022

(54) MELANOPHILIN ANTISENSE OLIGONUCLEOTIDES

(71) Applicant: OLIPASS CORPORATION, Gyeonggi-do (KR)

(72) Inventors: Seon-Young HAN, Gangwon-do (KR); Seokjoon HWANG, Gyeonggi-do (KR); Ji Eun KIM, Gyeonggi-do (KR); Kiho SUNG, Gyeonggi-do (KR); Yeonwoong LEE, Gyeonggi-do (KR)

(21) Appl. No.: 17/626,981

(22) PCT Filed: Jul. 14, 2020
(86) PCT No.: PCT/KR2020/009228

§ 371 Date: Jan. 13, 2022

(30) Foreign Application Priority Data
Jul. 18, 2019 (KR) 10-2019-0087228

(51) Int. Cl. C07K 14/00; C12N 15/113; A61K 8/64; A61K 8/60; A61Q 19/00

(52) U.S. Cl. CPC C07K 14/003; C12N 15/113; A61K 8/64; A61K 8/606; A61Q 19/00; C12N 2310/11; C12N 2310/3181; C12N 2320/33; A61K 38/00

(57) ABSTRACT

The present invention provides the peptide nucleic acid derivative which targets 3′ splice site of the human MLPH pre-mRNA “exon 2”. The peptide nucleic acid derivatives in the present invention strongly induce splice variants of the human MLPH mRNA in cell and are very useful to treat diseases or conditions of skin pigmentation associated with the human MLPH protein.

### TECHNICAL FIELD

This invention relates to peptide nucleic acid derivatives complementarily targeting the human melanophilin pre-mRNA for improvement of skin pigmentation mediated by melanophilin.

### BACKGROUND ART

Due to rapid population aging and the improvement of economy level, there has been a growing interest in quality of life and healthy aging. Especially, skin aging and pigmentation have received considerable attention since the signs of aging are most visible in the skin. As the prevention and treatment for skin aging is very important in quality of life, attention has been increasingly focused on related health food, cosmetics, medicine, and so on. There are two primary skin aging processes. One is intrinsic or natural aging accompanied aging and the other is extrinsic aging, which is caused by exogenous origin such as solar exposure, smoking, and malnutrition.

Skin Whitener: Skin whitener plays a major role in antiaging cosmetics market, which grows very rapidly. A number of skin whiteners have been developed based on various mechanisms of melanin biosynthesis in the skin, among which tyrosinase was the most representative target. A variety of skin whitening products have been developed, however, there have been some argues in safety, functionality, and specification and analysis method, and some of them are suspected to be harmful [Int. J. Cosmetic Sci. vol 33, 210-221 (2011); Int. J. Mol. Sci. vol 10, 2440-2475 (2009); Phytother Res. vol 21, 805-816 (2007)]. The active ingredient Rhododenol® of skin whitening cosmetics developed in Japan was recently reported to induce a skin disease such as vitiligo and the controversy over the safety issue of cosmetics products has been ongoing [Pigment Cell Melanoma Res. vol 27, 754-763 (2014)]. Therefore, it is necessary to develop efficacious and safe skin whitening products which are not related to melanin biosynthesis but target a de novo mechanism.

In addition, although there are a variety of tyrosinase inhibitors, their IC₅₀ values against tyrosinase are in most cases micromolar (μM) [J. Enzyme Inhibition Med. Chem. vol 32(1), 403-425 (2017)]. Such IC₅₀ values are considered to be poor considering their molecular sizes, and raise a possibility of cross reactivity with other molecular target(s). Since poor inhibitory activity is translated into large trans-dermal dose, the inhibitory potency needs to be markedly improved to meet the desired trans-dermal activity against skin pigmentation, and safety as well.

Melanophilin: A melanosome is an organelle 500 nm in diameter found in animal cells and is the site for synthesis, storage and transport of melanin, the most common light-absorbing pigment found in the animal kingdom. Melanosome is synthesized near the nucleus and moved to the end of the cell in melanocyte, which can be transported to nearby keratinocytes to induce pigmentation. In melanocyte melanosome is moved along the actin filament by a ternary complex of three proteins i.e. a link protein Rab27A which is attached to melanosome, a motor protein myosin-Va which is attached to actin filament, and a carrier protein melanophilin which connects Rab27A and myosin-Va [Kor. J. Aesthet. Cosmetol. vol 11, 417-426 (2013)]. Therefore, it would be possible to suppress the movement of melanosome to nearby keratinocytes and skin pigmentation by inhibiting the expression of these proteins.

Among three proteins, the inhibition of expressing Rab27A and myosin-Va were reported to induce immune and neural damage as well as hypopigmentation [Nat. Genet. vol 25 173-176 (2000); J. Cell Biol. vol 152, 835-842 (2001); J. Neurol. vol 247, 570-572 (2000)]. On the other hand, the inhibition of melanophilin, which is expressed only in melanocyte, was reported to induce hypopigmentation only without any other complications [J. Clin. Invest. vol 112, 450-456 (2003)]. Therefore, it is very interesting and necessary to develop products based on the mechanism of melanophilin expression for cosmetics or pharmaceuticals for excessive skin pigmentation, of which efficacy and safety are expected.

Pre-mRNA: Genetic information is carried on DNA (2-deoxyribose nucleic acid). DNA is transcribed to produce pre-mRNA (pre-messenger ribonucleic acid) in the nucleus. Mammalian pre-mRNA usually consists of exons and introns, and exon and intron are interconnected to each other as illustrated in FIG. 1a.

Splicing of Pre-mRNA: Pre-mRNA is processed into mRNA following deletion of introns by a series of complex reactions collectively called “splicing” which is schematically summarized in FIG. 1b [Ann. Rev. Biochem. 72(1), 291-336 (2003); Nature Rev. Mol. Cell Biol. 6(5), 386-398 (2005); Nature Rev. Mol. Cell Biol. 15(2), 108-121 (2014)].

Splicing is initiated by forming “spliceosome E complex” (i.e. early spliceosome complex) between pre-mRNA and splicing adapter factors. In “spliceosome E complex”, U1 binds to the junction of exon N and intron N, and U2AF³⁵ binds to the junction of intron N and exon (N+1). Thus the junctions of exon/intron or intron/exon are critical to the formation of the early spliceosome complex. “Spliceosome E complex” evolves into “spliceosome A complex” upon additional complexation with U2. The “spliceosome A complex” undergoes a series of complex reactions to delete or splice out the intron to adjoin the neighboring exons.

Ribosomal Protein Synthesis: Proteins are encoded by DNA (2-deoxyribose nucleic acid). In response to cellular stimulation or spontaneously, DNA is transcribed to produce pre-mRNA (pre-messenger ribonucleic acid) in the nucleus. The introns of pre-mRNA are enzymatically spliced out to yield mRNA (messenger ribonucleic acid), which is then translocated into the cytoplasm. In the cytoplasm, a complex of translational machinery called ribosome binds to mRNA and carries out the protein synthesis as it scans the genetic information encoded along the mRNA [Biochemistry, vol 41, 4503-4510 (2002); Cancer Res. vol 48, 2659-2668 (1988)].

Antisense Oligonucleotide (ASO): An oligonucleotide binding to nucleic acid including DNA, mRNA and pre-mRNA in a sequence specific manner (i.e. complementarily) is called antisense oligonucleotide (ASO).

If an ASO tightly binds to an mRNA in the cytoplasm, for example, the ASO may be able to inhibit the ribosomal protein synthesis along the mRNA. ASO needs to be present within the cytoplasm in order to inhibit the ribosomal protein synthesis of its target protein.

Antisense Inhibition of Splicing: If an ASO tightly binds to a pre-mRNA in the nucleus, the ASO may be able to inhibit or modulate the splicing of pre-mRNA into mRNA. ASO needs to be present within the nucleus in order to inhibit or modulate the splicing of pre-mRNA into mRNA. Such antisense inhibition of splicing produces an mRNA or mRNAs lacking the exon targeted by the ASO. Such mRNA(s) is called “splice variant(s)”, and encodes protein(s) smaller than the protein encoded by the full-length mRNA.

In principle, splicing can be interrupted by inhibiting the formation of “spliceosome E complex”. If an ASO tightly binds to a junction of (5′→3′) exon-intron, i.e. “5′ splice site”, the ASO blocks the complex formation between pre-mRNA and factor U1, and therefore the formation of “spliceosome E complex”. Likewise, “spliceosome E complex” cannot be formed if an ASO tightly binds to a junction of (5′→3′) intron-exon, i.e. “3′ splice site”.

3′ splice site and 5′ splice site are schematically illustrated in FIG. 1c.

Unnatural Oligonucleotides: DNA or RNA oligonucleotides are susceptible to degradation by endogenous nucleases, limiting their therapeutic utility. To date, many types of unnatural (naturally non-occurring) oligonucleotides have been developed and studied intensively [Clin. Exp. Pharmacol. Physiol. vol 33, 533-540 (2006)]. Some of them show extended metabolic stability compared to DNA and RNA. The chemical structures for a few of representative unnatural oligonucleotides are provided in FIG. 2a. Such oligonucleotides predictably bind to a complementary nucleic acid as DNA or RNA does.

Phosphorothioate Oligonucleotide: Phosphorothioate oligonucleotide (PTO) is a DNA analog with one of the backbone phosphate oxygen atoms replaced with a sulfur atom per monomer. Such a small structural change made PTO comparatively resistant to degradation by nucleases [Ann. Rev. Biochem. vol 54, 367-402 (1985)].

Reflecting the structural similarity in the backbone of PTO and DNA, they both poorly penetrate the cell membrane in most mammalian cell types. For some types of cells abundantly expressing transporter(s) of DNA, however, DNA and PTO show good cell penetration. Systemically administered PTOs are known to readily distribute to the liver and kidney [Nucleic Acids Res. vol 25, 3290-3296 (1997)].

In order to facilitate PTO's cell penetration in vitro, lipofection has been popularly practiced. However, lipofection physically alters the cell membrane, causes cytotoxicity, and therefore would not be ideal for long term in vivo therapeutic use.

Over the past 30 years, antisense PTOs and variants of PTOs have been clinically evaluated to treat cancers, immunological disorders, metabolic diseases, and so on [Biochemistry vol 41, 4503-4510 (2002); Clin. Exp. Pharmacol. Physiol. vol 33, 533-540 (2006)]. Many of such antisense drug candidates have not been successfully developed partly due to PTO's poor cell penetration. In order to overcome the poor cell penetration, PTO needs to be administered at high dose for therapeutic activity. However, PTOs are known to be associated with dose-limiting toxicity including increased coagulation time, complement activation, tubular nephropathy, Kupffer cell activation, and immune stimulation including splenomegaly, lymphoid hyperplasia, mononuclear cell infiltration [Clin. Exp. Pharmacol. Physiol. vol 33, 533-540 (2006)].

Many antisense PTOs have been found to show due clinical activity for diseases with a significant contribution from the liver or kidney. Mipomersen is a PTO analog which inhibits the synthesis of apoB-100, a protein involved in LDL cholesterol transport. Mipomersen manifested due clinical activity in atherosclerosis patients most likely due to its preferential distribution to the liver [Circulation vol 118(7), 743-753 (2008)]. ISIS-113715 is a PTO antisense analog inhibiting the synthesis of protein tyrosine phosphatase 1B (PTP1B), and was found to show therapeutic activity in type II diabetes patients. [Curr. Opin. Mol. Ther. vol 6, 331-336 (2004)].

Locked Nucleic Acid: In locked nucleic acid (LNA), the backbone ribose ring of RNA is structurally constrained to increase the binding affinity for RNA or DNA. Thus, LNA may be regarded as a high affinity DNA or RNA analog [Biochemistry vol 45, 7347-7355 (2006)].

Phosphorodiamidate Morpholino Oligonucleotide: In phosphorodiamidate morpholino oligonucleotide (PMO), the backbone phosphate and 2-deoxyribose of DNA are replaced with phosphoramidate and morpholine, respectively [Appl. Microbiol. Biotechnol. vol 71, 575-586 (2006)]. Whilst the DNA backbone is negatively charged, the PMO backbone is not charged. Thus the binding between PMO and mRNA is free of electrostatic repulsion between the backbones, and tends to be stronger than that between DNA and mRNA. Since PMO is structurally very different from DNA, PMO wouldn't be recognized by the hepatic transporter recognizing DNA. PMO may exhibit a different tissue distribution than PTO, but PMO, like PTO, doesn't readily penetrate the cell membrane.

Peptide Nucleic Acid: Peptide nucleic acid (PNA) is a polypeptide with N-(2-aminoethyl)glycine as the unit backbone, and was discovered by Dr. Nielsen and colleagues [Science vol 254, 1497-1500 (1991)]. The chemical structure and abbreviated nomenclature of PNA are illustrated in FIG. 2b. Like DNA and RNA, PNA also selectively binds to complementary nucleic acid. [Nature (London) vol 365, 566-568 (1992)]. In binding to complementary nucleic acid, the N-terminus of PNA is regarded as equivalent to the “5′-end” of DNA or RNA, and the C-terminus of PNA as equivalent to the “3′-end” of DNA or RNA.

Like PMO, the PNA backbone is not charged. Thus the binding between PNA and RNA tends to be stronger than the binding between DNA and RNA. Since PNA is markedly different from DNA in the chemical structure, PNA wouldn't be recognized by the hepatic transporter(s) recognizing DNA, and would show a tissue distribution profile different from that of DNA or PTO. However, PNA also poorly penetrates the mammalian cell membrane [Adv. Drug Delivery Rev. vol 55, 267-280 (2003)].

Modified Nucleobases to Improve Membrane Permeability of PNA: PNA was made highly permeable to mammalian cell membrane by introducing modified nucleobases with a cationic lipid or its equivalent covalently attached thereto. The chemical structures of such modified nucleobases are provided in FIG. 2c. Such modified nucleobases of cytosine, adenine, and guanine were found to predictably and complementarily hybridize with guanine, thymine, and cytosine, respectively [PCT Appl. No. PCT/KR2009/001256; EP2268607; U.S. Pat. No. 8,680,253].

Incorporation of such modified nucleobases onto PNA resembles situations of lipofection. By lipofection, oligonucleotide molecules with phosphate backbone are wrapped with cationic lipid molecules such as lipofectamine, and such lipofectamine/oligonucleotide complexes tend to penetrate membrane rather easily as compared to naked oligonucleotide molecules.

In addition to good membrane permeability, those PNA derivatives were found to possess ultra-strong affinity for complementary nucleic acid. For example, introduction of 4 to 5 modified nucleobases onto 11- to 13-mer PNA derivatives easily yielded a Tₘ gain of 20° C. or higher in duplex formation with complementary DNA. In addition, such PNA derivatives are highly sensitive to a single base mismatch. A single base mismatch resulted in a Tₘ loss of 11 to 22° C. depending on the type of modified base as well as PNA sequence.

Small Interfering RNA (siRNA): Small interfering RNA (siRNA) refers to a double stranded RNA of 20-25 base pairs [Microbiol. Mol. Biol. Rev. vol 67(4), 657-685 (2003)]. The antisense strand of siRNA somehow interacts with proteins to form an “RNA-induced Silencing Complex” (RISC). Then the RISC binds to a certain portion of mRNA complementary to the antisense strand of siRNA. The mRNA complexed with the RISC undergoes cleavage. Thus siRNA catalytically induces the cleavage of its target mRNA, and consequently inhibits the protein expression by the mRNA. The RISC does not always bind to the full complementary sequence within its target mRNA, which raises concerns relating to off-target effects of an siRNA therapy. Like other classes of oligonucleotide with DNA or RNA backbone, siRNA possesses poor cell permeability and therefore tends to show poor in vitro or in vivo therapeutic activity unless properly formulated or chemically modified to have good membrane permeability.

MLPH siRNA: The 21-mer siRNA targeting MLPH mRNA was reported to inhibit the expression of MLPH proteins in melan-a cell at 20 μM and to suppress the melanosome transport and enhance the aggregation of melanosome [Appl. Biochem. Biotechnol. Vol 172, 1882-1897 (2014)]. These results may be useful to the development of materials associated with the nelanophilin expression.

### DISCLOSURE OF THE INVENTION

### Problem to be Solved

A variety of skin whitening products based on various mechanisms of melanin biosynthesis have been developed, however, there have been some argues in safety, functionality, and specification and analysis method, and some of them are suspected to be harmful. Therefore, it is necessary to develop products based on the mechanisms not associated with melanin biosynthesis and to secure efficacy and safety. It is very interesting and necessary to develop products based on the mechanism of melanophilin expression for cosmetics or pharmaceuticals for excessive skin pigmentation, of which efficacy and safety are expected.

### Solution to the Problem

The present invention provides a peptide nucleic acid (PNA) derivative represented by Formula I, or a pharmaceutically acceptable salt thereof:

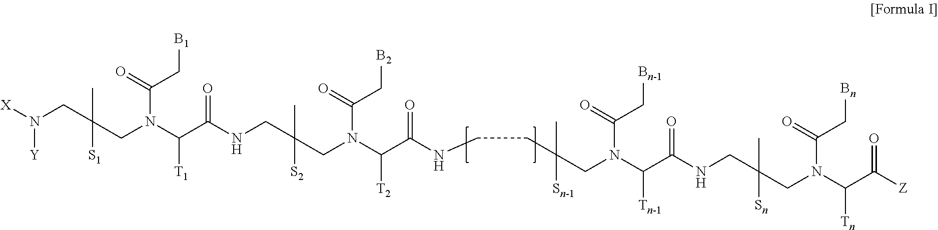

wherein,

n is an integer between 10 and 21;

the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;

the compound of Formula I is fully complementary to the human MLPH pre-mRNA, or partially complementary to the human MLPH pre-mRNA with one or two mismatches;

S₁, S₂, . . . , Sₙ₋₁, Sₙ, T₁, T₂, . . . , Tₙ₋₁, and Tₙ independently represent hydrido (—H), deuterido, substituted or non-substituted alkyl, or substituted or non-substituted aryl radical;

X and Y independently represent hydrido, deuterido, formyl [H—C(═O)—], aminocarbonyl [NH₂—C(═O)—], aminothiocarbonyl [NH₂—C(═S)—], substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;

Z represents hydrido, deuterido, hydroxy, substituted or non-substituted alkyloxy, substituted or non-substituted aryloxy, substituted or non-substituted amino, substituted or non-substituted alkyl, or substituted or non-substituted aryl radical;

B₁, B₂, . . . , Bₙ₋₁, and Bₙ are independently selected from natural nucleobases including adenine, thymine, guanine, cytosine and uracil, and unnatural nucleobases; and,

at least four of B₁, B₂, . . . , Bₙ₋₁, and Bₙ are independently selected from unnatural nucleobases with a substituted or non-substituted amino radical covalently linked to the nucleobase moiety.

The compound of Formula I induces the skipping of “exon 2” in the human MLPH pre-mRNA, yields the human MLPH mRNA splice variant(s) lacking “exon 2”, and is useful to cosmetics or pharmaceuticals for excessive skin pigmentation which is related to the activity of melanophilin proteins.

The condition that “n is an integer between 10 and 21” literally means that n is an integer selectable from a group of integers of 11, 12, 13, 14, 15, 16, 17, 18, 19, and 20.

The compound of Formula I complementarily binds to the 3′ splice site of “intron 1” and “exon 2” of the human MLPH pre-mRNA derived from the human MLPH DNA [NCBI Reference Sequence: NG_007286]. The 30-mer sequence of [(5′→3′) CCUGUGACAUUCCAGGUGUGACCCCGACAA] spans 3′ splice site of 15-mer “intron 1” and 15-mer “exon 2” in the human MLPH pre-mRNA. Thus the 30-mer pre-mRNA sequence may be conventionally denoted as [(5′→3′) ccugugacauuccag I GUGUGACCCCGACAA], wherein the intron and exon sequence are provided as “small” and “capital” letters, respectively, and the junction “intron 1” and “exon 2” is expressed with “|”.

The chemical structures of natural or unnatural nucleobases in the PNA derivative of Formula I are exemplified in FIG. 3. Natural (i.e. naturally occurring) or unnatural (naturally non-occurring) nucleobases of this invention comprise but are not limited to the nucleobases provided in FIG. 3. Provision of such unnatural nucleobases is to illustrate the diversity of allowable nucleobases, and therefore should not be interpreted to limit the scope of the present invention.

The substituents adopted to describe the PNA derivative of Formula I are exemplified in FIGS. 4a-4e. FIG. 4a provides examples for substituted or non-substituted alkyl radicals. Substituted or non-substituted alkylacyl and substituted or non-substituted arylacyl radicals are exemplified in FIG. 4b. FIG. 4c illustrates examples for substituted or non-substituted alkylamino, substituted or non-substituted arylamino, substituted or non-substituted aryl, substituted or non-substituted alkylsulfonyl or arylsulfonyl, and substituted or non-substituted alkylphosphonyl or arylphosphonyl radicals. FIG. 4d provides examples for substituted or non-substituted alkyloxycarbonyl or aryloxycarbonyl, substituted or non-substituted alkyl aminocarbonyl or arylaminocarbonyl radicals. In FIG. 4e are provided examples for substituted or non-substituted alkylaminothiocarbonyl, substituted or non-substituted arylaminothiocarbonyl, substituted or non-substituted alkyloxythiocarbonyl, and substituted or non-substituted aryloxythiocarbonyl radicals. Provision of such exemplary substituents is to illustrate the diversity of allowable substituents, and therefore should not be interpreted to limit the scope of the present invention. A skilled person in the field may easily figure out that oligonucleotide sequence is the overriding factor for sequence specific binding of oligonucleotide to the target pre-mRNA sequence over substituents in the N-terminus or C-terminus.

The compound of Formula I tightly binds to the complementary DNA as exemplified in the prior art [PCT/KR2009/001256]. The duplex between the PNA derivative of Formula I and its full-length complementary DNA or RNA possesses a Tₘ value too high to be reliably determined in aqueous buffer. The PNA compound of Formula I yields high Tₘ values with complementary DNAs of shorter length.

The compound of Formula I tightly binds to the target 3′ splice site of the human MLPH pre-mRNA transcribed from the human MLPH gene, and interferes with the formation of “spliceosome early complex” to yield MLPH mRNA splice variant(s) lacking “exon 2” (exon 2 skipping).

The strong RNA affinity allows the compound of Formula I to induce the skipping of MLPH “exon 2”, even when the PNA derivative possesses one or two mismatches with the target 3′ splice site in the MLPH pre-mRNA. Similarly the PNA derivative of Formula I may still induce the skipping of MLPH “exon 2” in a MLPH mutant pre-mRNA possessing one or two SNPs (single nucleotide polymorphism) in the target splice site.

The compound of Formula I possesses good cell permeability and can be readily delivered into cell as “naked” oligonucleotide as exemplified in the prior art [PCT/KR2009/001256]. Thus the compound of this invention induces the skipping of “exon 2” in the human MLPH pre-mRNA, and yields human MLPH mRNA splice variant(s) lacking MLPH “exon 2” in cells treated with the compound of Formula I as “naked” oligonucleotide. The compound of Formula I does not require any means or formulations for delivery into cell to potently induce the skipping of the target exon in cells. The compound of Formula I readily induces the skipping of “exon 2” in the human MLPH pre-mRNA of cells treated with the compound of this invention as “naked” oligonucleotide at sub-femtomolar concentration.

Owing to the good cell or membrane permeability, the PNA derivative of Formula I can be topically administered as “naked” oligonucleotide to induce the skipping of MLPH “exon 2” in the skin. The compound of Formula I does not require a formulation to increase trans-dermal delivery into target tissue for the intended therapeutic or biological activity. Usually the compound of Formula I is dissolved in water and co-solvent, and topically or trans-dermally administered at sub-picomolar concentration to elicit the desired therapeutic or biological activity in target skin. The compound of this invention does not need to be heavily or invasively formulated to elicit the topical therapeutic activity. Nevertheless, the PNA derivative of Formula I can be formulated with cosmetic ingredients or adjuvants as topical cream or lotion. Such topical cosmetic cream or lotion may be useful to improve skin pigmentation.

The compound of Formula I of the present invention can be topically administered to a subject at a therapeutically or biologically effective concentration ranging from 1 aM to higher than 1 nM, which would vary depending on the dosing schedule, conditions or situations of the subject, and so on.

The compound (PNA derivative) of Formula I can be variously formulated including but not limited to injections, nasal spray, transdermal patch, and so on. In addition, the PNA derivative of Formula I can be administered to the subject at therapeutically effective dose and the dose of administration can be diversified depending on indication, administration route, dosing schedule, conditions or situations of the subject, and so on.

The compound of Formula I may be used as combined with a pharmaceutically acceptable acid or base including but not limited to sodium hydroxide, potassium hydroxide, hydrochloric acid, methanesulfonic acid, citric acid, trifluoroacetic acid, and so on.

The PNA derivative of Formula I or a pharmaceutically acceptable salt thereof can be administered to a subject in combination with a pharmaceutically acceptable adjuvant including but not limited to citric acid, hydrochloric acid, tartaric acid, stearic acid, polyethyleneglycol, polypropyleneglycol, ethanol, isopropanol, sodium bicarbonate, distilled water, preservative(s), and so on.

Of interest is a PNA derivative of Formula I, or a pharmaceutically acceptable salt thereof:

wherein,

n is an integer between 10 and 21;

the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;

the compound of Formula I is fully complementary to the human MLPH pre-mRNA, or partially complementary to the human MLPH pre-mRNA with one or two mismatches;

S₁, S₂, . . . , Sₙ₋₁, Sₙ, T₁, T₂, . . . , Tₙ₋₁, and Tₙ independently represent hydrido, deuterido radical;

X and Y independently represent hydrido, deuterido, formyl, aminocarbonyl, aminothiocarbonyl, substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;

Z represents hydrido, hydroxy, substituted or non-substituted alkyloxy, substituted or non-substituted aryloxy, or substituted or non-substituted amino radical;

B₁, B₂, . . . , Bₙ₋₁, and Bₙ are independently selected from natural nucleobases including adenine, thymine, guanine, cytosine and uracil, and unnatural nucleobases;
at least four of B₁, B₂, . . . , Bₙ₋₁, and Bₙ are independently selected from unnatural nucleobases represented by Formula II, Formula III, or Formula IV:

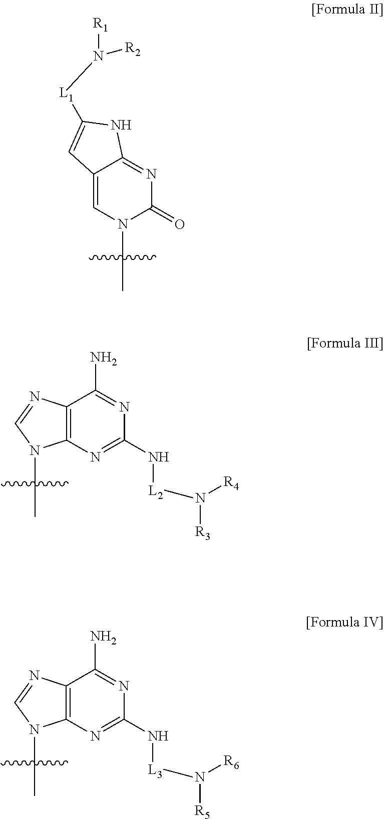

wherein,

R₁, R₂, R₃, R₄, R₅ and R₆ are independently selected from hydrido and substituted or non-substituted alkyl radical;

L₁, L₂ and L₃ are a covalent linker represented by Formula V covalently linking the basic amino group to the nucleobase moiety:

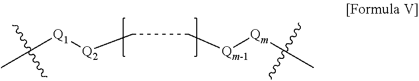

wherein,

Q₁ and Qₘ are substituted or non-substituted methylene radical [—CH₂—, —CH(substituent)-, —C(substituent)₂-], and Qₘ is directly linked to the basic amino group;

Q₂, Q₃, . . . , and Qₘ₋₁ are independently selected from substituted or non-substituted methylene, oxygen (—O—), sulfur (—S—), and substituted or non-substituted amino radical [—N(H)—, or —N(substituent)-]; and,

m is an integer between 1 and 15.

Of high interest is a PNA oligomer of Formula I, or a pharmaceutically acceptable salt thereof:

wherein,

n is an integer between 11 and 19;

the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;

the compound of Formula I is fully complementary to the human MLPH pre-mRNA;

S₁, S₂, . . . , Sₙ₋₁, Sₙ, T₁, T₂, . . . , Tₙ₋₁, and Tₙ are hydrido radical;

X and Y independently represent hydrido, substituted or non-substituted alkylacyl, or substituted or non-substituted alkyloxycarbonyl radical;

Z represents substituted or non-substituted amino radical;

B₁, B₂, . . . , Bₙ₋₁, and Bₙ are independently selected from natural nucleobases including adenine, thymine, guanine, cytosine and uracil, and unnatural nucleobases;

at least five of B₁, B₂, . . . , Bₙ₋₁, and Bₙ are independently selected from unnatural nucleobases represented by Formula II, Formula III, or Formula IV;

R₁, R₂, R₃, R₄, R₅ and R₆ are hydrido radical;

Q₁ and Qₘ , are methylene radical, and Qₘ is directly linked to the basic amino group;

Q₂, Q₃, . . . , and Qₘ₋₁ are independently selected from methylene and oxygen radical; and,

m is an integer between 1 and 9.

Of higher interest is a PNA derivative of Formula I, or a pharmaceutically acceptable salt thereof:

wherein,

n is an integer between 11 and 19;

the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;

the compound of Formula I is fully complementary to the human MLPH pre-mRNA;

S₁, S₂, . . . , Sₙ₋₁, Sₙ, T₁, T₂, . . . , Tₙ₋₁, and Tₙ are hydrido radical;

X is hydrido radical;

Y represents substituted or non-substituted alkyloxycarbonyl radical;

Z represents substituted or non-substituted amino radical;

B₁, B₂, . . . , Bₙ₋₁, and Bₙ are independently selected from natural nucleobases including adenine, thymine, guanine, cytosine and uracil, and unnatural nucleobases;

at least five of B₁, B₂, . . . , Bₙ₋₁, and Bₙ are independently selected from unnatural nucleobases represented by Formula II, Formula III, or Formula IV;

R₁, R₂, R₃, R₄, R₅ and R₆ are hydrido radical;

L₁ represents —(CH₂)₂—O—(CH₂)₂—, —CH₂—O—(CH₂)₂—, —CH₂—O—(CH₂)₃—, —CH₂—O—(CH₂)₄—, or —CH₂—O—(CH₂)₅—; and,

L₂ and L₃ are independently selected from —(CH₂)₂—O—(CH₂)₂—, —(CH₂)₃—O—(CH₂)₂—, —(CH₂)₂—O—(CH₂)₃—, —(CH₂)₂—, —(CH₂)₃—, —(CH₂)₄—, —(CH₂)₅—, —(CH₂)₆—, —(CH₂)₇—, and —(CH₂)₈—.

Of specific interest is a PNA derivative of the present invention which is selected from the group of compounds provided below (Hereinafter referred to as ASOs 1, 6, 7, and 8, respectively), or a pharmaceutically acceptable salt thereof:

(N→C)Fethoc-GG(5)T-CA(6)C-A(6)C(1O2)C-TG(5)G-
A(6)A-NH₂;
(N→C)Fethoc-C(1O2)GG(6)-GG(6)T-CA(5)C-
A(5)C(1O2)C-TG(6)G-A(5)A-NH₂;
(N→C)Fethoc-GG(6)G-G(6)TC-A(5)CA(5)-C(1O2)CT-
G(6)GA(5)-ATG(6)-NH₂;
and
(N→C)Fethoc-GG(6)T-CA(5)C-A(5)C(1O2)C-TG(6)G-
A(5)AT-G(6)TC(1O2)-NH₂;

wherein,

A, T, G and C are PNA monomers with a natural nucleobase of adenine, thymine, guanine and cytosine, respectively;

C(pOq), A(p), and G(p) are PNA monomers with an unnatural nucleobase represented by Formula VI, Formula VII, and Formula VIII, respectively;

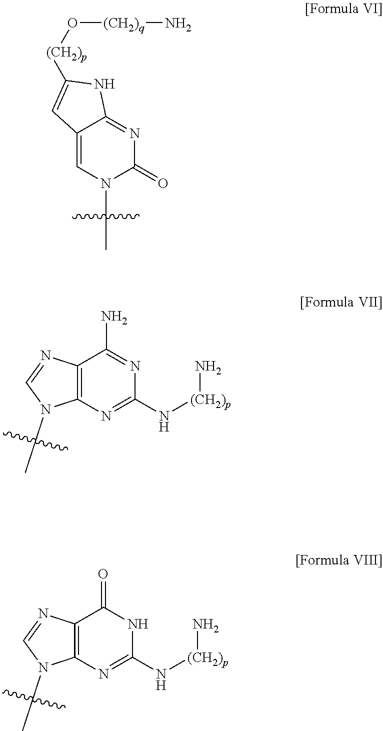

wherein,

p and q are integers, for example, in the case of ASO 1, p is 1, 5, or 6 and q is 2; and,

“Fethoc-” is the abbreviation for “[2-(9-fluorenyl)ethyl-1-oxy]carbonyl” and “—NH₂” is for non-substituted “-amino” group.

FIG. 5 collectively and unambiguously provides the chemical structures for the PNA monomers abbreviated as A, G, T, C, C(pOq), A(p), A(pOq), G(p) and G(pOq). As discussed in the prior art [PCT/KR2009/001256], C(pOq) is regarded as a “modified cytosine” PNA monomer due to its hybridization for “guanine”. A(p) is taken as “modified adenine” PNA monomers due to their hybridization for “thymine”, and G(p) is taken as “modified guanine” PNA monomers due to their hybridization for “cytosine”. In addition, in order to illustrate the abbreviations employed for such PNA derivatives, the chemical structure of 14-mer ASO 1 “(N→C) Fethoc-GG(5)T-CA(6)C-A(6)C(1O2)C-TG(5)G-A(6)A-NH₂” is unambiguously provided in FIG. 6.

ASO 1 is equivalent to the DNA sequence of “(5′→3′) GGT-CAC-ACC-TGG-AA” for complementary binding to pre-mRNA. The 14-mer PNA has a 14-mer complementary overlap with the marked “bold” and “underlined” RNA sequence of

[(5′ → 3′)
ccugugacauuccag | GUGUGACCCCGACAA]

spanning the junction of “intron 1” and “exon 2” in the human MLPH pre-mRNA.

In some embodiments, the present invention provides a method of treating diseases or conditions associated with human MLPH gene transcription in a subject, comprising administering to the subject the peptide nucleic acid derivative of the present invention or a pharmaceutically acceptable salt thereof.

In some embodiments, the present invention provides a method of treating skin pigmentation in a subject, comprising administering to the subject the peptide nucleic acid derivative of the present invention or a pharmaceutically acceptable salt thereof.

In some embodiments, the present invention provides a pharmaceutical composition for treating diseases or conditions associated with human MLPH gene transcription, comprising the peptide nucleic acid derivative of the present invention or a pharmaceutically acceptable salt thereof.

In some embodiments, the present invention provides a cosmetic composition for treating diseases or conditions associated with human MLPH gene transcription, comprising the peptide nucleic acid derivative of the present invention or a pharmaceutically acceptable salt thereof.

In some embodiments, the present invention provides a pharmaceutical composition for treating skin pigmentation, comprising the peptide nucleic acid derivative of the present invention or a pharmaceutically acceptable salt thereof.

In some embodiments, the present invention provides a cosmetic composition for treating skin pigmentation, comprising the peptide nucleic acid derivative of the present invention or a pharmaceutically acceptable salt thereof.

### Effect of Invention

Diseases or conditions associated with human MLPH gene transcription can be treated by administering a PNA derivative of Formula I or a pharmaceutically acceptable salt thereof.

Excessive skin pigmentation can be treated by administering a PNA derivative of Formula I or a pharmaceutically acceptable salt thereof.

### BRIEF EXPLANATION OF DRAWINGS

FIG. 1a. Illustration of the pre-mRNA structure.

FIG. 1b. Schematic illustration of splicing process for intron N removal.

FIG. 1c. Schematic illustration of 3′ splice site and 5′ splice site in spliceosome E complex.

FIG. 2a. Chemical structures for DNA and representative unnatural oligonucleotides.

FIG. 2b. The chemical structure and abbreviated nomenclature of prototype PNA.

FIG. 2c. Modified nucleobases developed to improve the membrane permeability of PNA.

FIGS. 3a-3c. Examples of natural or unnatural (modified) nucleobases selectable for the peptide nucleic acid derivative of Formula I.

FIG. 4a. Examples of substituents selectable for the peptide nucleic acid derivative of Formula I, substituted or non-substituted alkyls.

FIG. 4b. Examples of substituents selectable for the peptide nucleic acid derivative of Formula I, substituted or non-substituted alkylacyls, and substituted or non-substituted arylacyls.

FIG. 4c. Examples of substituents selectable for the peptide nucleic acid derivative of Formula I, substituted alkylaminos, substituted arylaminos, substituted or non-substituted aryls, substituted or non-substituted alkylsulfonyls, substituted or non-substituted arylsulfonyls, substituted or non-substituted alkylphosphonyls, and substituted or non-substituted arylsulfonyls.

FIG. 4d. Examples of substituents selectable for the peptide nucleic acid derivative of Formula I, substituted or non-substituted alkyloxycarbonyls and substituted or non-substituted aryloxycarbonyls, substituted or non-substituted alkylaminocarbonyls, and substituted or non-substituted arylaminocarbonyls.

FIG. 4e. Examples of substituents selectable for the peptide nucleic acid derivative of Formula I, substituted or non-substituted alkyloxythiocarbonyls and substituted or non-substituted alkylaminothiocarbonyls, substituted or non-substituted arylaminothiocarbonyls, and substituted or non-substituted aryoxythiocarbonyls.

FIG. 5. Chemical structures of abbreviated PNA monomers, A, G, T, C, C(pOq), A(p), A(pOq), G(p), and G(pOq).

FIG. 6. Chemical structure of abbreviated 14-mer “(N→C) Fethoc-GG(5)T-CA(6)C-A(6)C(1O2)C-TG(5)G-A(6)A-NH₂”.

FIG. 7. Chemical structures of Fmoc-PNA monomers used to synthesize the PNA derivatives of this invention.

FIG. 8. Schematic illustration of a typical monomer elongation cycle adopted in SPPS of this invention.

FIG. 9a. C₁₈-reverse phase HPLC chromatogram for “ASO 2” before HPLC purification.

FIG. 9b. C₁₈-reverse phase HPLC chromatogram for “ASO 2” after HPLC purification.

FIG. 10. ES-TOF mass spectral data obtained with “ASO 2” after HPLC purification.

FIG. 11a-11d. Real-time qPCR data in melanoma melan-a treated with “ASO 2”, “ASO 3”, “ASO 4”, and “ASO 5”.

FIG. 12a-12c. Electrophoretic analysis data in melanoma melan-a treated with “ASO 3”, “ASO 4”, and “ASO 5”.

FIG. 13a-13d. Western blot data in melanoma melan-a treated with “ASO 2”, “ASO 3”, “ASO 4”, and “ASO 5”.

FIG. 14a. Microscope digital images for the evaluation of melanosome aggregation levels in melanoma melan-a treated with siRNA and ASOs.

FIG. 14b. Quantified melanosome aggregation levels.

FIG. 15. Real-time qPCR data in human melanocytes treated with “ASO 1”.

FIG. 16. Western blot data in human melanocytes treated with “ASO 1”.

FIG. 17. Microscope digital images for the evaluation of melanosome aggregation levels and relative melanosome aggregation level in human melanocytes treated with “ASO 1”.

FIG. 18. Relative survival rates in human melanocytes treated with “ASO 1”.

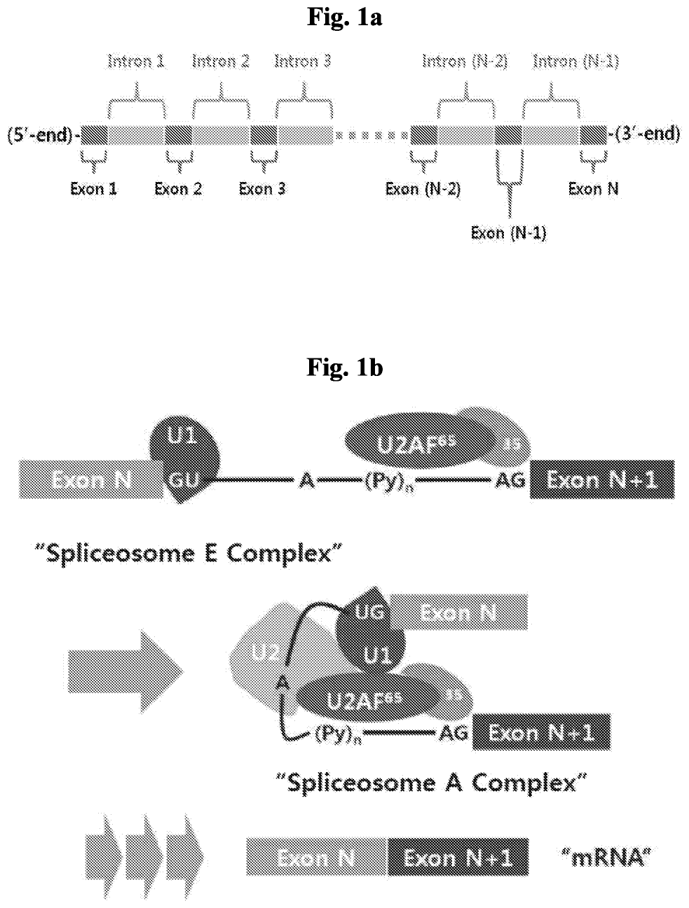

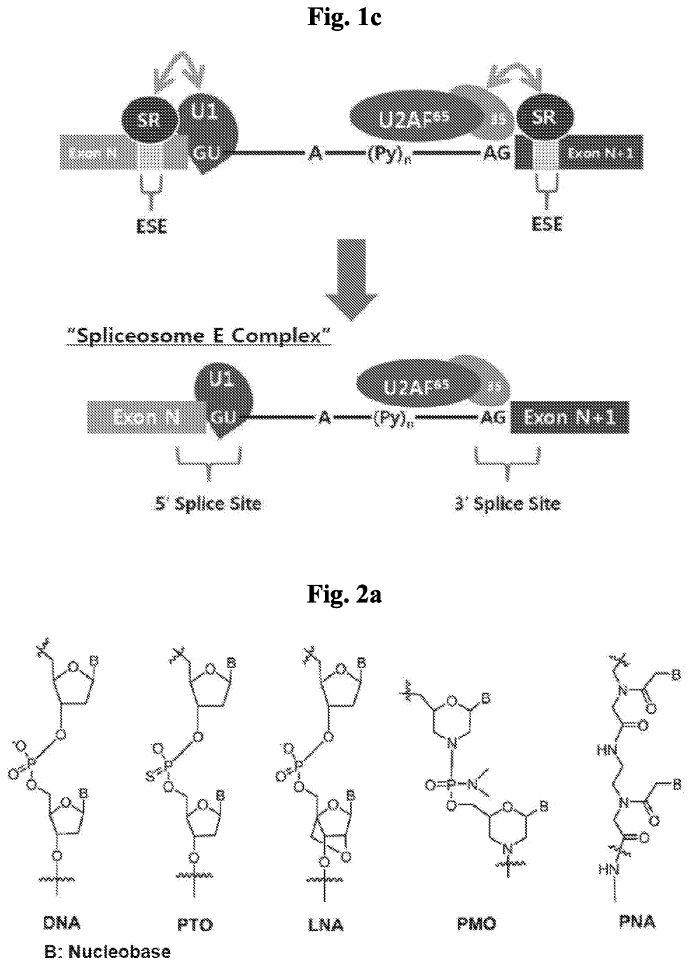

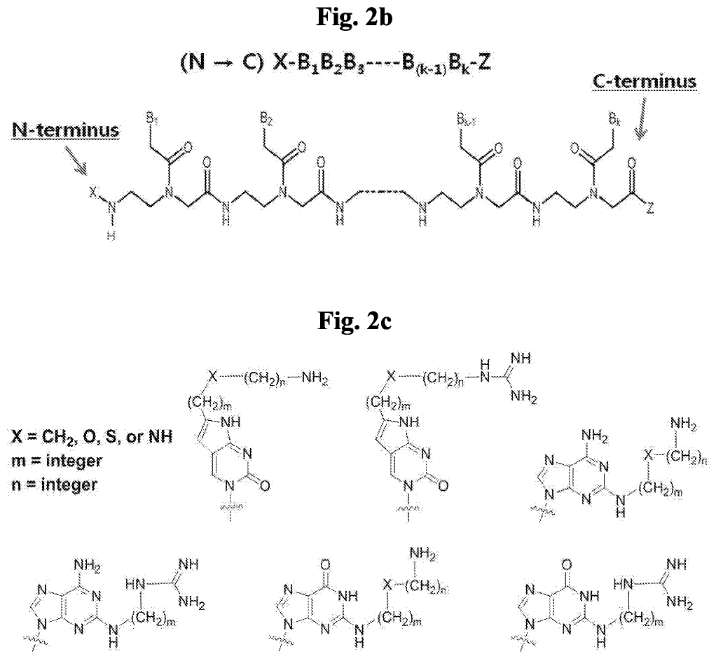

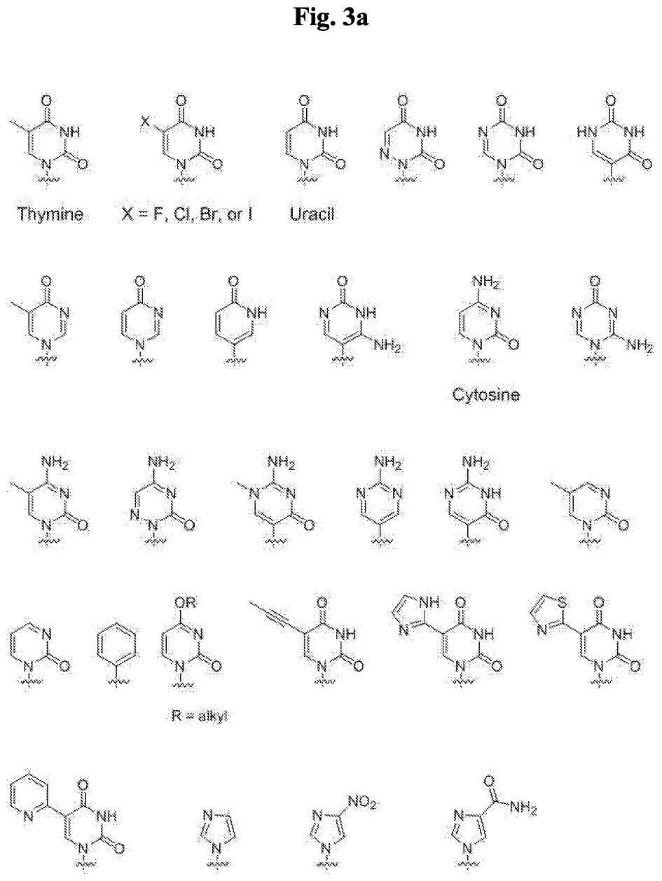

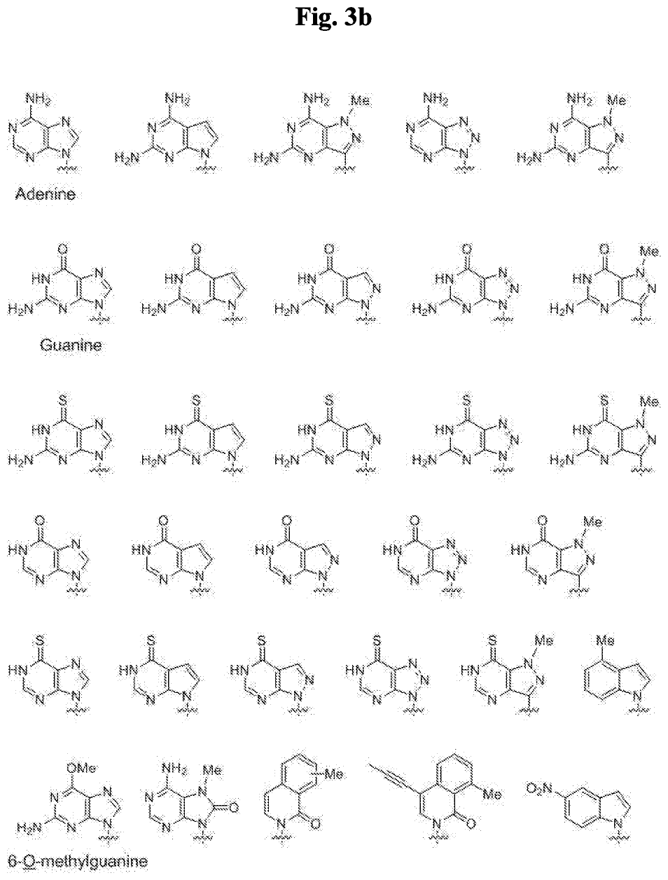

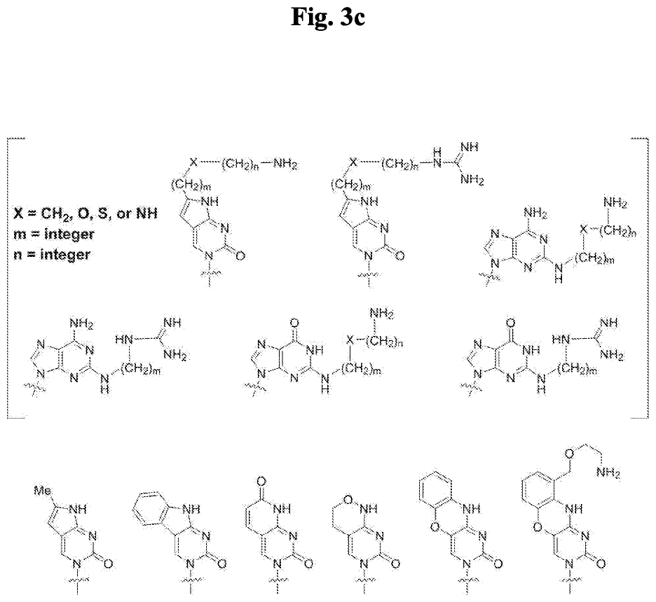

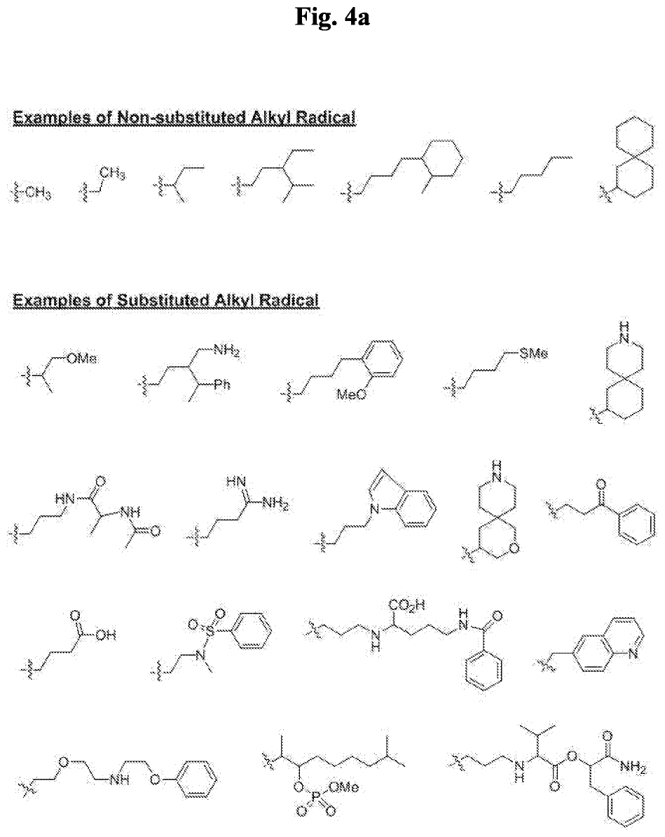

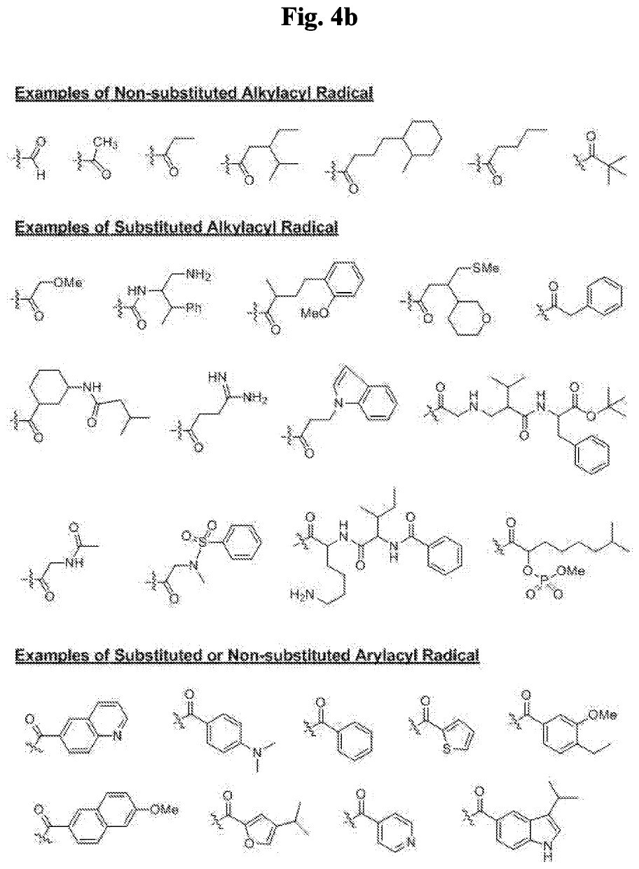

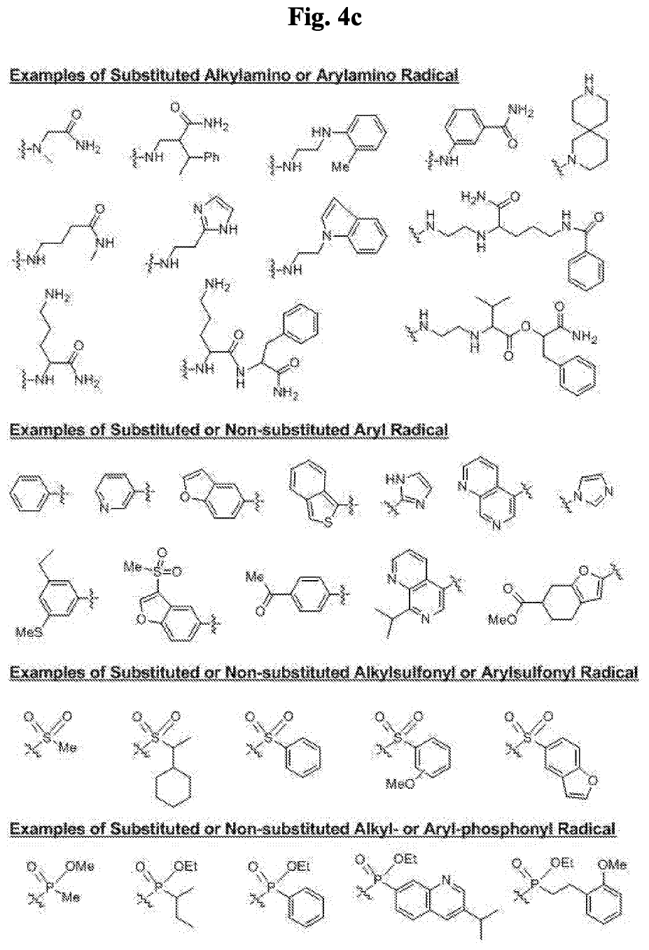

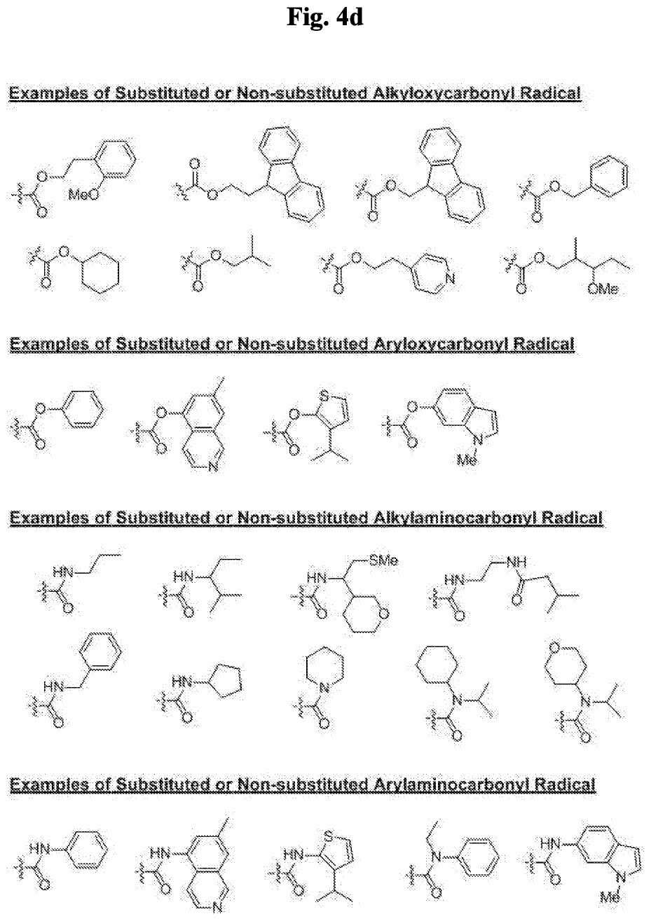

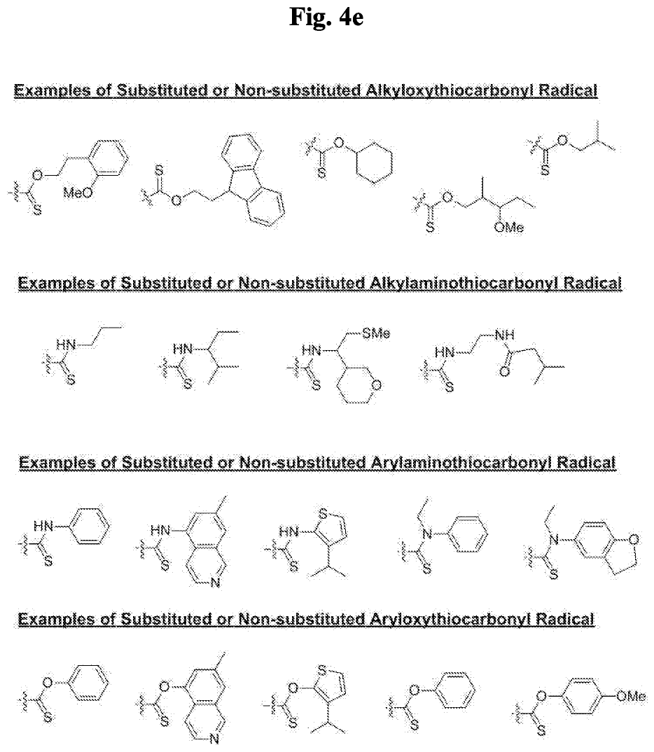

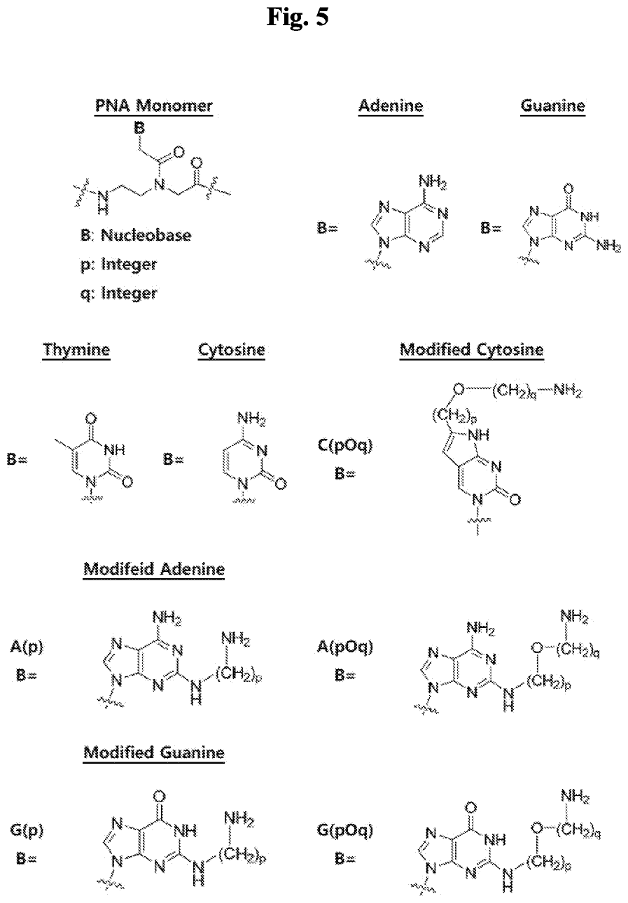

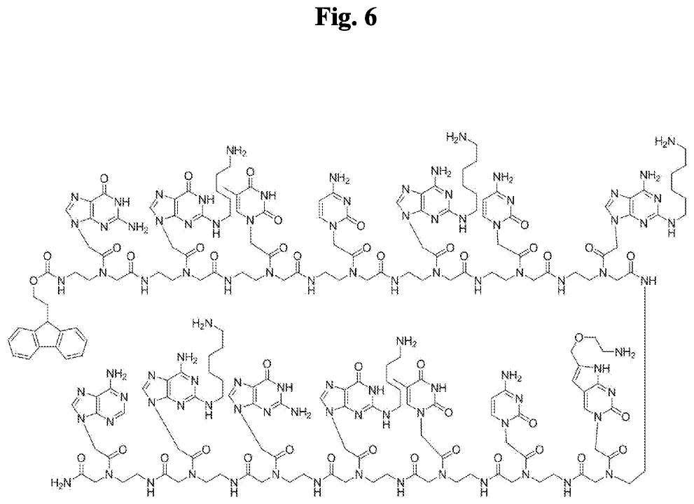

### BEST MODE FOR CARRYING OUT THE INVENTION

### General Procedures for Preparation of PNA Oligomers

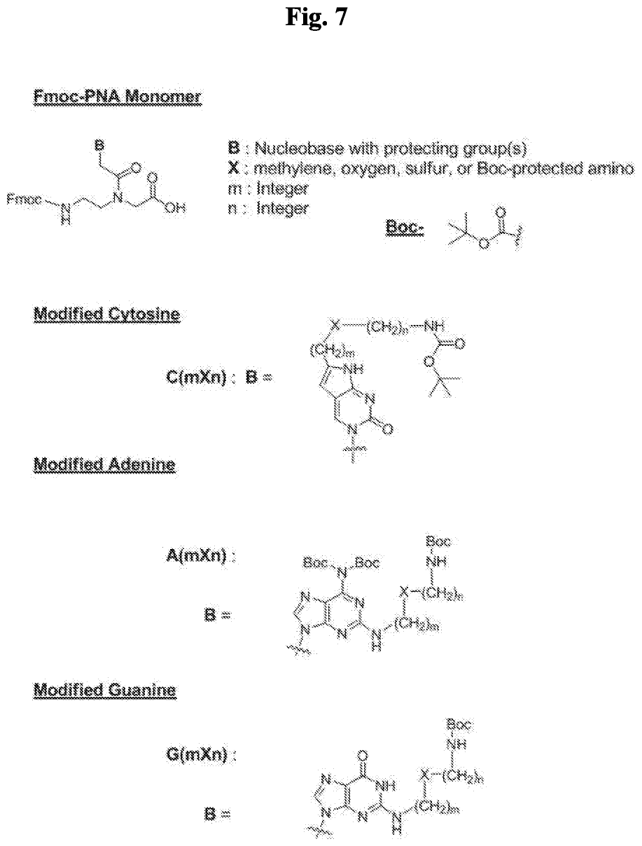

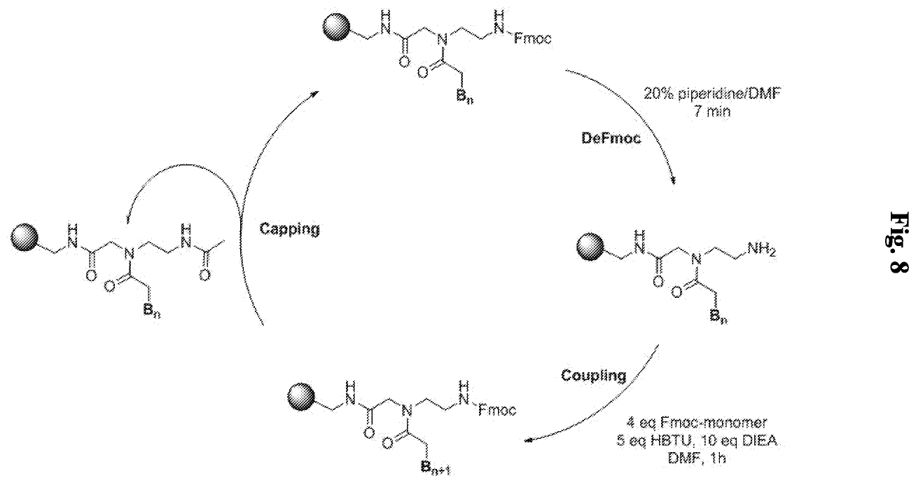

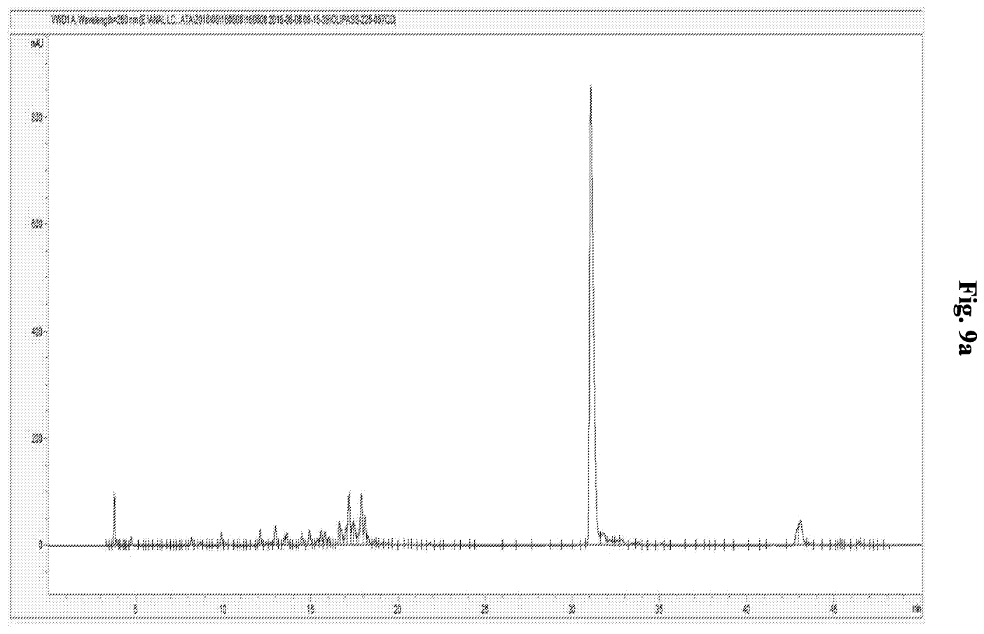

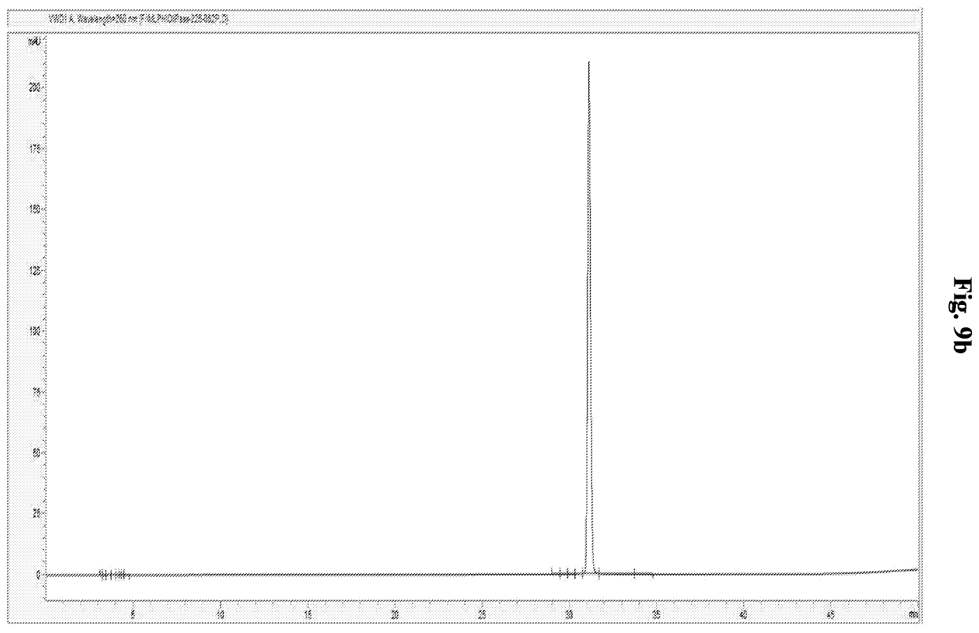

PNA monomers with a modified nucleobase were synthesized as described in the prior art [PCT/KR 2009/001256] or with minor modifications. Chemical structures of Fmoc-PNA monomers used to synthesize the PNA derivatives of this invention are provided in FIG. 7. Fmoc-PNA [Fmoc={(9-fluorenyl)methoxy}carbonyl] monomers in FIG. 7 should be taken as examples, and therefore should not be taken to limit the scope of the present invention. Such Fmoc-PNA monomers with a modified nucleobase and Fmoc-PNA monomers with a naturally occurring nucleobase were used to synthesize the PNA oligomers by solid phase peptide synthesis (SPPS) as provided in FIG. 8 based on Fmoc-chemistry according to the method disclosed in the prior art [U.S. Pat. No. 6,133,444; WO96/40685] with minor but due modifications. The solid support employed in this study was H-Rink Amide-ChemMatrix resin purchased from PCAS BioMatrix Inc. (Quebec, Canada). PNA oligomers were purified by C₁₈-reverse phase HPLC (water/acetonitrile or water/methanol with 0.1% TFA) and characterized by mass spectrometry including ESI/TOF/MS. FIGS. 9a and 9b are exemplary HPLC chromatograms for “ASO 2” before and after HPLC purification, respectively. FIG. 10 is ESI/TOF/MS spectrum of “ASO 2” after HPLC purification, which should be taken as examples for oligomers, and therefore should not be taken to limit the scope of the present invention.

FIG. 8 illustrates a typical monomer elongation cycle adopted in the SPPS of this invention, and each reaction step is briefly provided as follows. [Activation of H-Rink-ChemMatrix Resin] When the amine on the resin was not protected with Fmoc, 0.01 mmol (ca 20 mg resin) of the ChemMatrix resin in 1.5 mL 20% piperidine/DMF was vortexed in a libra tube for 20 min, and the DeFmoc solution was filtered off. The resin was washed for 30 sec each in series with 1.5 mL methylene chloride (MC), 1.5 mL dimethylformamide (DMF), 1.5 mL MC, 1.5 mL DMF, and 1.5 mL MC. The resulting free amines on the solid support were subjected to coupling either with an Fmoc-PNA monomer.

[DeFmoc] When the amine on the resin was protected with Fmoc, the suspension of 0.01 mmol (ca 20 mg) of the resin in 1.5 mL 20% piperidine/DMF was vortexed for 7 min, and the DeFmoc solution was filtered off. The resin was washed for 30 sec each in series with 1.5 mL MC, 1.5 mL DMF, 1.5 mL MC, 1.5 mL DMF, and 1.5 mL MC. The resulting free amines on the solid support were immediately subjected to coupling with an Fmoc-PNA monomer.

[Coupling with Fmoc-PNA Monomer] The free amines on the solid support were coupled with an Fmoc-PNA monomer as follows. 0.04 mmol of PNA monomer, 0.05 mmol HBTU, and 0.1 mmol DIEA were incubated for 2 min in 1 mL anhydrous DMF, and added to the resin with free amines. The resin solution was vortexed for 1 hour and the reaction medium was filtered off. Then the resin was washed for 30 sec each in series with 1.5 mL MC, 1.5 mL DMF, and 1.5 mL MC.

[Capping] Following the coupling reaction, the unreacted free amines were capped by shaking for 5 min in 1.5 mL capping solution (5% acetic anhydride and 6% 2,6-leutidine in DMF). Then the capping solution was filtered off and washed for 30 sec each in series with 1.5 mL MC, 1.5 mL DMF, and 1.5 mL MC.

[Introduction of “Fethoc-” Radical in N-Terminus] “Fethoc-” radical was introduced to the N-terminus by reacting the free amine on the resin with “Fethoc-OSu” by the following method. The suspension of the resin in the solution of 0.1 mmol of Fethoc-OSu and 0.1 mmol

DIEA in 1 mL anhydrous DMF was vortexed for 1 hr, and the solution was filtered off. The resin was washed for 30 sec each in series with 1.5 mL MC, 1.5 mL DMF, and 1.5 mL MC. The chemical structure of “Fethoc-OSu” [CAS No. 179337-69-0, C₂₀H₁₇NO₅, MW 351.36] used in the present invention is provided as follows.

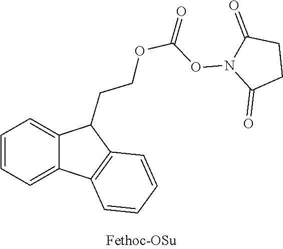 [SMILES: O=C(OCCC1c2ccccc2-c2ccccc21)ON1C(=O)CCC1=O]

[Cleavage from Resin] PNA oligomers bound to the resin were cleaved from the resin by shaking for 3 hours in 1.5 mL cleavage solution (2.5% tri-isopropylsilane and 2.5% water in trifluoroacetic acid). The resin was filtered off and the filtrate was concentrated under reduced pressure. The resulting residue was triturated with diethyl ether and the resulting precipitate was collected by filtration for purification by reverse phase HPLC.

[HPLC Analysis and Purification] Following a cleavage from resin, the crude product of a PNA derivative was purified by C₁₈-reverse phase HPLC eluting water/acetonitrile or water/methanol (gradient method) containing 0.1% TFA. FIGS. 9a and 9b are exemplary HPLC chromatograms for “ASO 2” before and after HPLC purification, respectively.

### Synthetic Examples for PNA Derivative of Formula I

In order to complementarily target the 3′ splice site of “exon 2” in the human MLPH pre-mRNA, PNA derivatives of this invention were prepared according to the synthetic procedures provided above or with minor modifications. Provision of such PNA derivatives targeting the human MLPH pre-mRNA is to exemplify the PNA derivatives of Formula I, and should not be interpreted to limit the scope of the present invention.

TABLE 1
PNA derivatives complementarily targeting the 3′ splice site of
“exon 2” in the human MLPH pre-mRNA along with structural
characterization data by mass spectrometry.

| PNA | | Exact Mass, m/z | |
|---|---|---|---|
| Example | PNA Sequence (N→C) | theor.^{a} | obs.^{b} |
| ASO 1 | Fethoc-GG(5)T-CA(6)C-A(6)C(1O2)C-TG(5)G-A(6)A- | 4664.183 | 4664.193 |
| | NH₂ | | |
| ASO 6 | Fethoc-C(1O2)GG(6)-GG(6)T-CA(5)C-A(5)C(1O2)C- | 5679.643 | 5679.673 |
| | TG(6)G-A(5)A-NH₂ | | |
| ASO 7 | Fethoc-GG(6)G-G(6)TC-A(5)CA(5)-C(1O2)CT- | 5987.803 | 5987.870 |
| | G(6)GA(5)-ATG(6)-NH₂ | | |
| ASO 8 | Fethoc-GG(6)T-CA(5)C-A(5)C(1O2)C-TG(6)G- | 5920.738 | 5920.798 |
| | A(5)AT-G(6)TC(1O2)-NH₂ | | |

^{a}theoretical exact mass,
^{b}observed exact mass

Table 1 provides PNA derivatives complementarily targeting the 3′ splice site of “exon 2” in the human MLPH pre-mRNA read out from the human MLPH gene [NCBI Reference Sequence: NG_007286] along with structural characterization data by mass spectrometry. Provision of the peptide nucleic acid derivatives of the present invention in Table 1 is to exemplify the PNA derivatives of Formula I, and should not be interpreted to limit the scope of the present invention.

“ASO 1” has a 14-mer complementary overlap with the 14-mer sequence marked “bold” and “underlined” within the 30-mer RNA sequence of

[(5′ → 3′)
ccugugacauuccag | GUGUGACCCCGACAA]

spanning the junction of “intron 1” and “exon 2” in the human MLPH pre-mRNA. Thus “ASO 1” possesses a 6-mer overlap with “intron 1” and an 8-mer overlap with “exon 2” within the human MLPH pre-mRNA.

### Synthetic Examples for PNA Derivatives Complementarily Targeting the Mouse MLPH pre-mRNA

In order to facilitate the efficacy evaluation with readily available melan-a from mice, PNA derivatives of this invention complementarily targeting the 3′ splice site spanning the junction of “intron 1” and “exon 2” in the mouse MLPH pre-mRNA read out from the mouse MLPH gene [NCBI Reference Sequence: NC_000067] were prepared. The 30-mer sequence of [(5′→3′) CCUGUGACUUUCUAGGUGUGGCCUGGAUGA] spans 3′ splice site of 15-mer “intron 1” and 15-mer “exon 2” in the mouse MLPH pre-mRNA. Thus the 30-mer pre-mRNA sequence may be conventionally denoted as [(5′→3′) ccugugacuuucuag|GUGUGGCCUGGAUGA], wherein the intron and exon sequence are provided as “small” and “capital” letters, respectively, and the junction “intron 1” and “exon 2” is expressed with “|”.

Provision of such PNA derivatives targeting the mouse MLPH pre-mRNA is to exemplify the PNA derivatives of Formula I, and should not be interpreted to limit the scope of the present invention.

TABLE 2
PNA derivatives complementarily targeting the 3′ splice site of
“exon 2” in the mouse MLPH pre-mRNA along with structural
characterization data by mass spectrometry.

| PNA | | Exact Mass, m/z | |
|---|---|---|---|
| Example | PNA Sequence (N→C) | theor.^{a} | obs.^{b} |
| ASO 2 | Fethoc-GG(5)C-CA(6)C-A(6)C(1O2)C-TA(6)G-A(6)A- | 4662.215 | 4662.225 |
| | NH₂ | | |
| ASO 3 | Fethoc-C(12)CA(5)-GG(6)C-CA(5)C-A(5)C(1O2)C- | 5594.637 | 5594.510 |
| | TA(5)G-A(5)A-NH₂ | | |
| ASO 4 | Fethoc-GG(6)C-CA(5)C-A(5)C(1O2)C-TA(5)G- | 5900.745 | 5900.555 |
| | A(5)AA(5)-GTC(1O2)-NH₂ | | |
| ASO 5 | Fethoc-CA(5)G-G(6)CC-A(5)CA(5)-C(1O2)CT- | 5902.819 | 5912.324 |
| | A(5)GA(5)-AA(5)G-NH₂ | | |

^{a}theoretical exact mass,
^{b}observed exact mass

Table 2 provides PNA derivatives complementarily targeting the 3′ splice site of “exon 2” in the mouse MLPH pre-mRNA read out from the mouse MLPH gene along with structural characterization data by mass spectrometry. Provision of the peptide nucleic acid derivatives of the present invention in Table 2 is to exemplify the PNA derivatives of Formula I, and should not be interpreted to limit the scope of the present invention.

“ASO 3” has a 17-mer complementary overlap with the sequence marked “bold” and “underlined” within the following RNA sequence of

[(5′ → 3′)
ccugugacuuucuag | GUGUGGCCUGGAUGA]

spanning the junction of “intron 1” and “exon 2” in the mouse MLPH pre-mRNA. Thus “ASO 3” possesses a 6-mer overlap with “intron 1” and an 11-mer overlap with “exon 2” within the mouse MLPH pre-mRNA.

### Binding Affinity of “ASO” for Complementary DNA

The PNA derivatives of Formula I were evaluated for their binding affinity for 10-mer DNAs complementarily targeting either the N-terminal or C-terminal. The binding affinity was assessed by Tₘ value for the duplex between PNA and 10-mer complementary DNA. The duplex between PNA derivatives and fully complementary DNAs show Tₘ values too high to be reliably determined in aqueous buffer solution, since the buffer solution tends to boil during the Tₘ measurement. Tₘ values for full length PNAs can be predicted and compared based on the Tₘ value for the duplex between PNA and 10-mer complementary DNA.

Tₘ values were determined on a UV/Vis spectrometer as follows. A mixed solution of 320 μL, of 50 μM PNA oligomer, 320 μL of 50 μM complementary 10-mer DNA, and 3.36 mL of aqueous buffer (pH 7.16, 10 mM sodium phosphate, 100 mM NaCl) in 15 mL polypropylene falcon tube was incubated at 90° C. for a few minute and slowly cooled down to ambient temperature. Then the solution was transferred into a 3 mL quartz UV cuvette equipped with an air-tight cap, and the cuvette was mounted on an Agilent 8453 UV/Visible spectrophotometer. The absorbance changes at 260 nm were recorded with increasing the temperature of the cuvette by either 0.5 or 1° C. per minute. From the absorbance vs temperature curve, the temperature showing the largest increase rate in absorbance was read out as the Tₘ between PNA and 10-mer DNA. The DNAs for Tₘ measurement were purchased from Bioneer (www.bioneer.com, Dajeon, Republic of Korea) and used without further purification.

Observed Tₘ values of the PNA derivatives of Formula I are very high and the result is provided in Table 3.

TABLE 3
Tₘ values between PNAs and 10-mer complementary DNA targeting
either the N-terminal or the C-terminal of PNA.

Tₘ Value, ° C.

| PNA | 10-mer DNA against | 10-mer DNA against |
|---|---|---|
| Example | N-Terminal | C-Terminal |
| ASO 1 | 81.02 | 90.37 |
| ASO 6 | 76.07 | 86.47 |
| ASO 7 | 85.47 | 77.22 |
| ASO 8 | 83.52 | 73.12 |

For example, “ASO 1” showed a Tₘ value of 81.02° C. for the duplex with the 10-mer complementary DNA targeting the N-terminal 10-mer in the PNA as marked “bold” and “underlined” in

[(N → C)Fethoc-GG(5)T-CA(6)C-A(6)C(1O2)C-TG(5)G-
A(6)A-NH₂].

In the meantime, “ASO 1” showed a Tₘ of 90.37° C. for the duplex with the 10-mer complementary DNA targeting the C-terminal 10-mer in the PNA as marked “bold” and “underlined” in

[(N → C)GG(5)T-CA(6)C-A(6)C(1O2)C-TG(5)G-A(6)A-NH₂].

### EXAMPLES FOR BIOLOGICAL ACTIVITIES OF PNA DERIVATIVES OF FORMULA I

PNA derivatives in this invention were evaluated for in vitro MLPH antisense activities in mouse melanoma melan-a and human melanocyte by use of real-time quantitative polymerase chain reaction (RT-qPCR) and so on. The biological examples were provided as examples to illustrate the biological profiles of the PNA derivatives of Formula I, and therefore should not be interpreted to limit the scope of the current invention.

### Example 1. Effects of ASOs on MLPH Expression in Mouse Melanoma Melan-a

“ASO 2”, “ASO 3”, “ASO 4”, and “ASO 5” were evaluated for their ability to affect MLPH expression in mouse melanoma melan-a as described below.

[Cell Culture & ASO Treatment] Mouse melanoma melan-a were grown in RPMI 1640 (GIBCO, Cat. No.11875-093) supplemented with 10% FBS (Fetal Bovine Serum) (Cat. No. 10099-41, GIBCO), 1% streptomycin/penicillin (Cat. No. 15140-122, GIBCO), and 200 nM TPA (Sigma, Cat. No. 79346) under 5% CO₂ atmosphere at 37° C. Mouse melanoma melan-a (2×10⁵) were grown in 60 mm culture dish for 24 hours for stabilization, and were treated either with nothing (negative control) or with “ASO 2”, “ASO 3”, “ASO 4”, or “ASO 5” for 48 hours at 100 zM, 10 aM, 1 fM, or 1 μM.

[RNA Extraction & cDNA synthesis] Total RNA was extracted using RNeasy Mini kit (Qiagen, Cat. No. 714106) according to the manufacturer's instructions from ASOs treated cells and cDNA was prepared from 500 ng of RNA by use of PrimeScript™ 1^{st} strand cDNA Synthesis kit (Takara, Cat. No. 6110A). To a mixture of 500 ng of RNA, 1 microliter of random hexamer, and 1 microliter of dNTP (10 mM) in PCR tube was added DEPC-treated water to a total volume of 10 microliter, which was reacted at 65° C. for 5 minutes. cDNA was synthesized by adding 10 microliter of PrimeScript RTase reaction mixture and reacting at 30° C. for 10 minutes and at 42° C. for 60 minutes, successively.

[Real-Time qPCR] In order to evaluate the expression level of mouse MLPH mRNA real-time qPCR was performed with synthesized cDNA by use of Taqman probe. The mixture of cDNA, Taqman probe (Thermo, Cat. No. Mm00453498_m1), IQ supermix (BioRad, Cat. No. 170-8862) and nuclease free water in PCR tube was under reaction by use of CFX96 Touch Real-Time system (BioRad) according to the cycle conditions specified as follows: at 95° C. for 3 min followed by 40 cycles of 10 sec at 95° C. and 30 sec at 60° C. Fluorescence intensity was measured at the end of every cycle and the result of PCR was evaluated by the melting curve. After the threshold cycle (Ct) of each gene was standardized by that of GAPDH, the relative expression level of mouse MLPH mRNA was compared and analyzed.

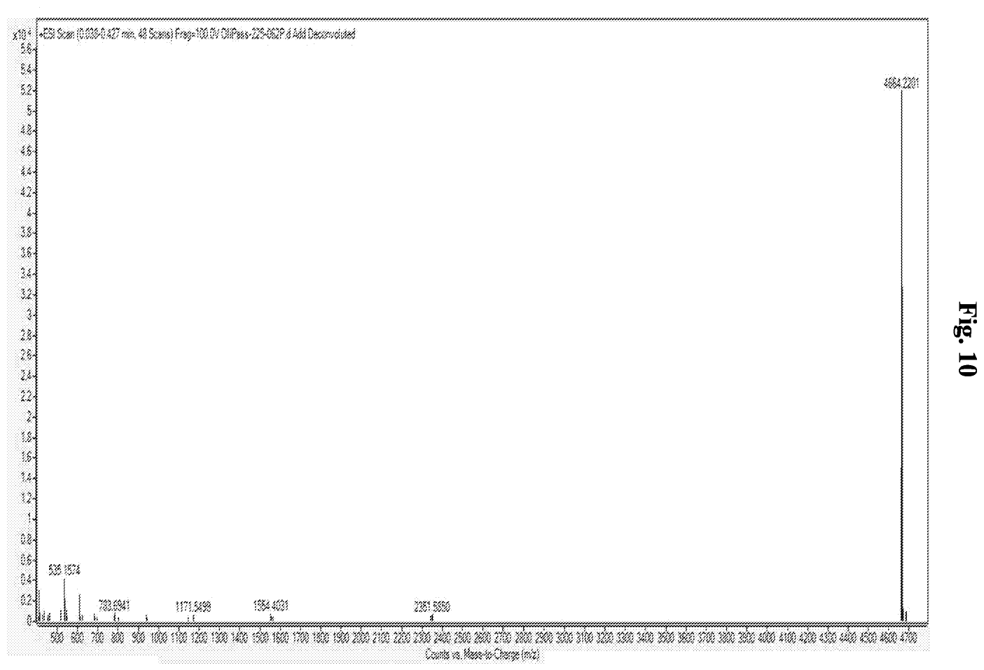

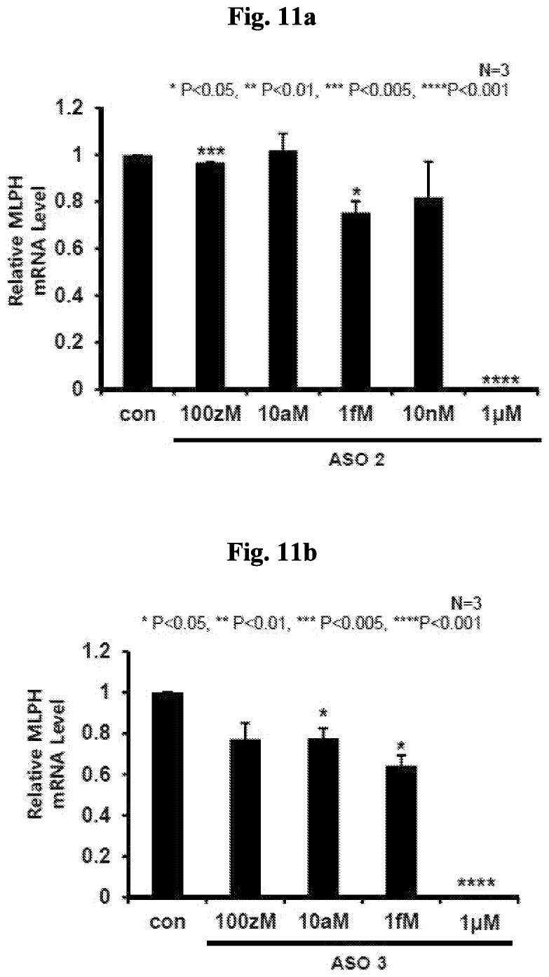

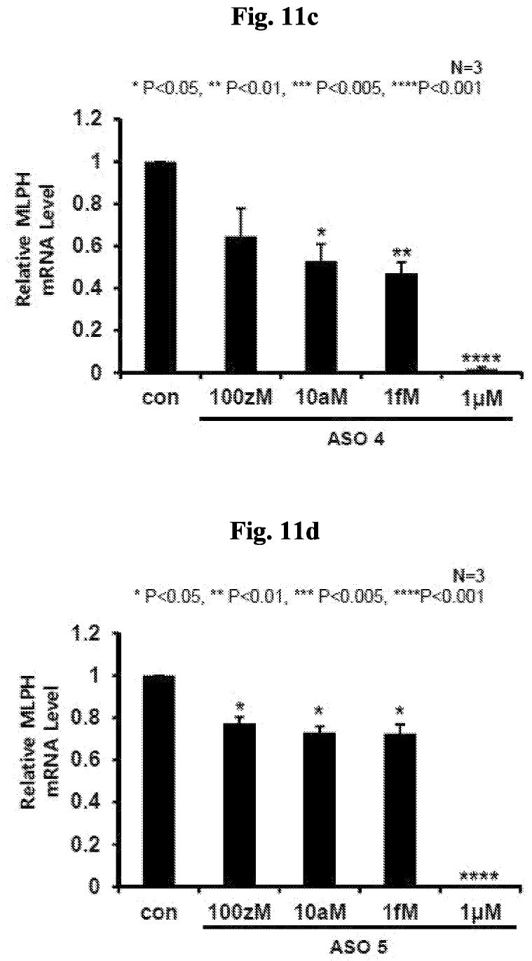

FIG. 11a, FIG. 11b, FIG. 11c, and FIG. 11d provide the relative expression levels of mouse MLPH mRNA in “ASO 2”, “ASO 3”, “ASO 4”, and “ASO 5” treated cells, respectively. The relative expression levels of mouse MLPH mRNA in “ASO 3”, “ASO 4”, and “ASO 5” (not “ASO 2”) treated cells were reduced in a dose dependent manner. (Student T-test was done to check the statistical significance of the findings)

[Exon Skipping] In order to evaluate the exon skipping level of mouse MLPH mRNA standard PCR was performed with synthesized cDNA by use of PCR PreMix (Bioneer, Cat. No. K-2612, South Korea) against a set of primers of [forward: (5′→3′) TAG CTC AGT GCA CCC TGA CA; and reverse: (5′→3′) GAG AGA CCG GAT CAC TTT GG] according to the following cycle conditions: 95° C. for 5 min followed by 25 cycles of 1 min at 95° C. , 1 min at 59° C., 2 min at 72° C., and an additional 3 min at 72° C.

The PCR products (10 microliter) were subjected to electrophoretic separation on a 2% agarose gel. The target bands were collected and analyzed by Sanger Sequencing to evaluate exon skipping sequence.

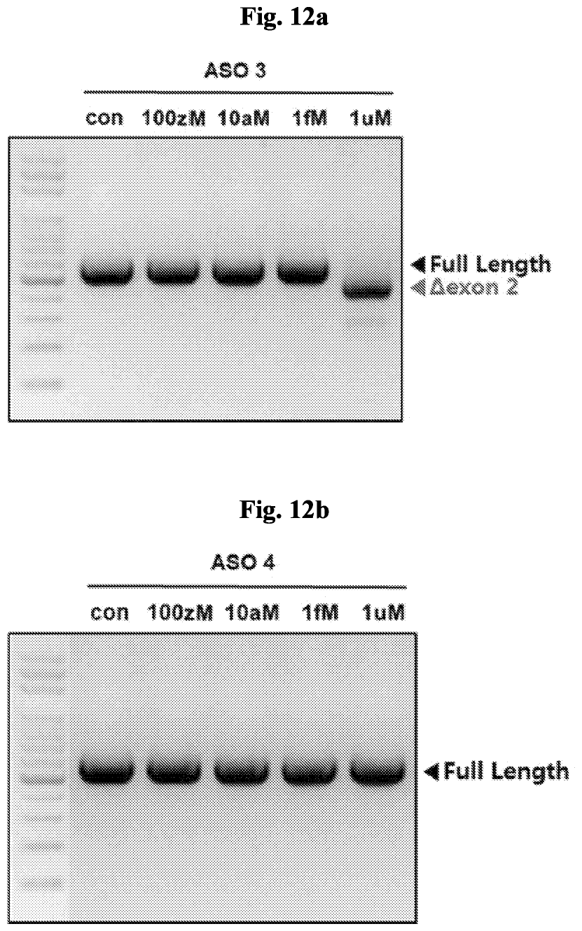

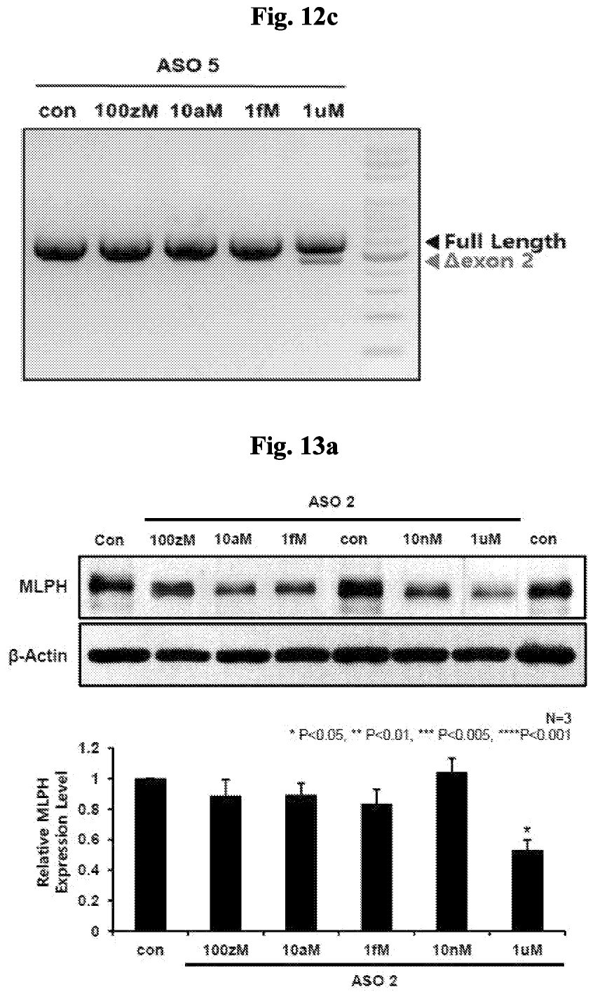

FIG. 12a, FIG. 12b, and FIG. 12c provide the results of electrophoretic separation in “ASO 3”, “ASO 4”, and “ASO 5” treated cells, respectively. While the cells treated with “ASO 4” did not yield the exon skipping band and the cells treated with 1 μM “ASO 5” faintly yielded the exon 2 skipping band, the cells treated with 1 μM “ASO 3” yielded the exon 2 skipping band only instead of full length MLPH mRNA band. Thus “ASO 3” targets MLPH pre-mRNA at 3′ splice site and yields exon 2 skipped splice variant MLPH mRNA at 1 μM.

[Western Blotting] Cells were grown as above. After 48 hours from being treated with each ASOs, the cells were collected, and then washed 2 times with cold PBS (phosphate buffered saline) and dissolved in RIPA buffer (Cell Signaling, Cat. No. 9806). The protein was quantified with BCA solution (Thermo, Cat. No. 23225) and purified by 10% SDS-PAGE Gel. The protein was transferred on PVDF membrane (polyvinylidene fluoride membrane) (Millipore, Cat. No. IPVH00010), which was blocked in 5% skim milk buffer solution for 1 hour. The membrane was probed with an anti-MLPH (Proteintech, Cat. No. 10338-1AP) and anti-β-actin (Sigma, Cat. No. A3854) as a primary antibody, and goat anti-rabbit (CST, Cat. No. 7074) was used as a secondary antibody. HRP substrate (Millipore, Cat. No. WBKLS0500) was utilized for the detection of chemiluminescent signal and the signal intensity was measured by using Gel Doc system (ATTO). Based on Western blotting results of each bands, the relative expression levels of MLPH were quantified with Image-J program and converted to graph.

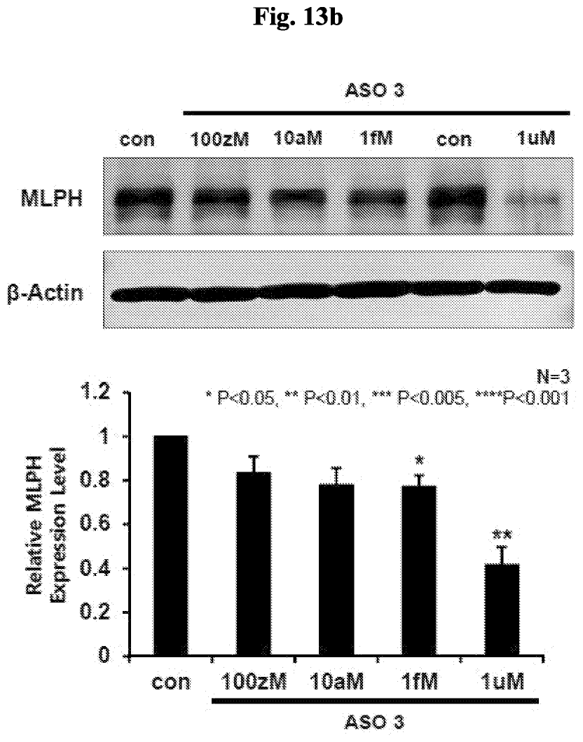

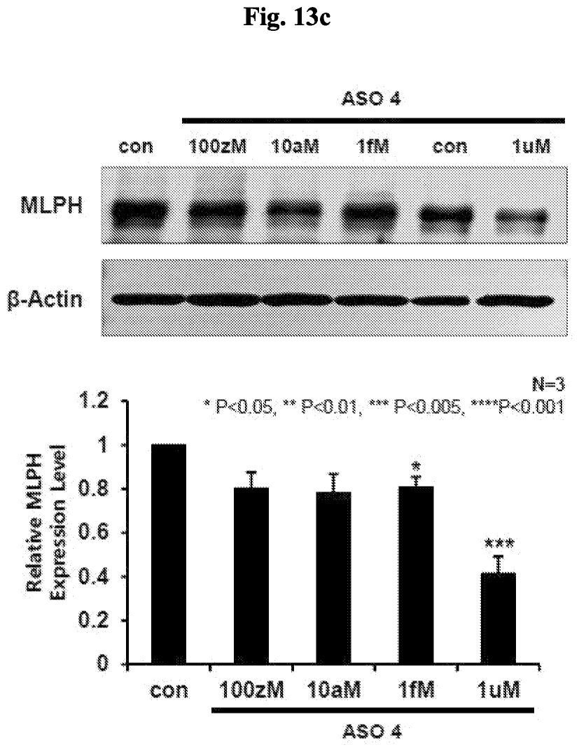

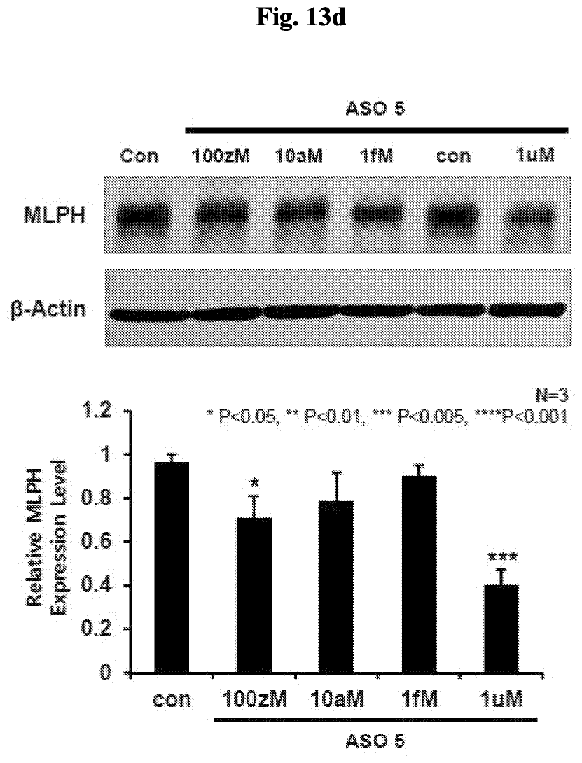

FIG. 13a, FIG. 13b, FIG. 13c, and FIG. 13d provide the relative expression levels of mouse MLPH protein in “ASO 2”, “ASO 3”, “ASO 4”, and “ASO 5” treated cells, respectively. Overall in ASO treated cells the relative expression levels of mouse MLPH protein were reduced and especially in “ASO 3” treated cells the levels were reduced in a dose dependent manner. (Student T-test was done to check the statistical significance of the findings)

[Melanosome Aggregation] Cells were grown as above. In order to evaluate the degree of melanosome aggregation melanophilin siRNA was used as a positive control. Melanophilin siRNA was purchased from Bioneer in Daejeon of South Korea, which has a sense sequence of (5′→3′) GGGCAAAAUACAAAAGGAG and an antisense sequence of (5′→3′) 5′-CUCCUUUUGUAUUUUGCCC-3′. Melanoma melan-a was grown in 60 mm culture dish for 24 hours and the medium was changed to 3 mL of Opti-MEM (Gibco, Cat. No. 31985-070). To a mixture of 500 μL of Opti-MEM and 5 μL of lipofectamin2000 (Invitrogen, Cat.No.11668-019) in 1.5 mL eppendorf tube was added 3 mL of 10 μM siRNA stock solution (final concentration of siRNA: 10 nM). After 15 min, the resulting solution was added to culture dish in 3 mL of Opti-MEM and cultured at 37° C. for 6 hours, to which new culture medium was applied.

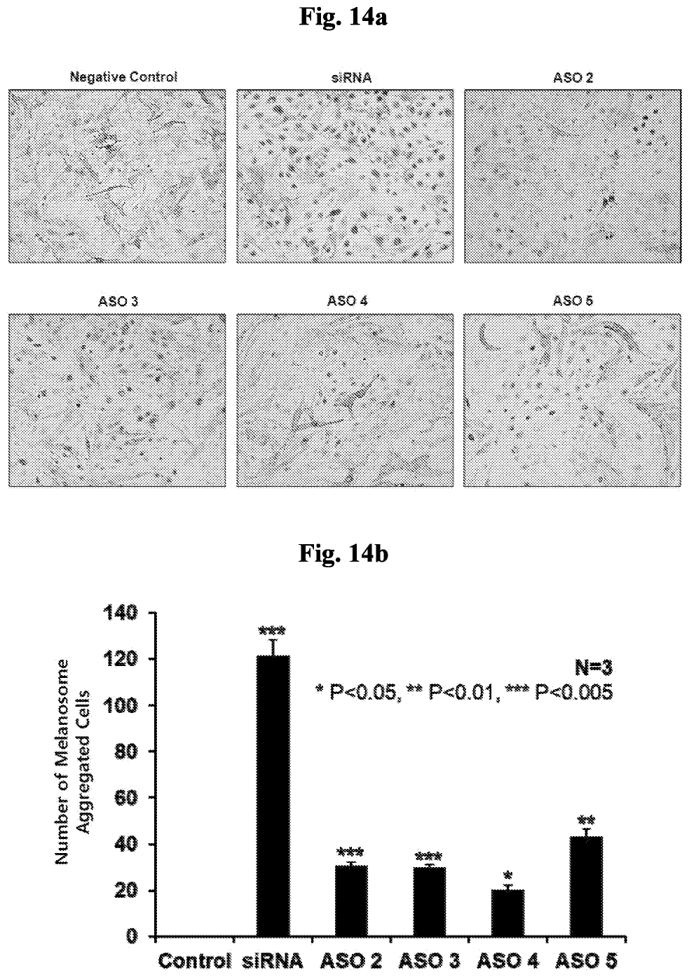

On 24 or 48 hours after treatment of siRNA or ASOs, in order to evaluate the degree of melanosome aggregation the cells were photographed with bright-field microscope in two orders of magnitude. FIG. 14a provides microscope digital image for negative control, siRNA treated cell, and ASO treated cell to evaluate the degree of melanosome aggregation and FIG. 14b provides the number of melanosome aggregated cells.

As can be seen in FIGS. 14a and 14b, cells treated with siRNA or ASOs for 24 or 48 hours yielded more melanosome aggregation compared to the negative control, which can be interpreted that melanophilin siRNA or ASOs inhibited the expression of MLPH proteins and to suppress the melanosome transport and enhance the aggregation of melanosome. Thus melanophilin siRNA or ASOs is expected to inhibit skin pigmentation by suppressing the melanosome transport.

### Example 2. Effects of ASOs on MLPH Expression in Human Melanocyte

“ASO 1” was evaluated for their ability to affect MLPH expression in human melanocyte as described below.

[Cell Culture & ASO Treatment] Human melanocyte (Lonza, Cat. No. CC-2586) were grown in melanocyte dedicated medium (Lonza, Cat. No. CC-3249) supplemented with 1% streptomycin/penicillin (GIBCO, Cat. No. 15140-122) under 5% CO₂ atmosphere at 37° C. Human melanocyte (3×10⁵) were grown in 60 mm culture dish for 24 hours for stabilization, and were treated either with nothing (negative control) or with “ASO 1” for 24 or 48 hours at 1 μM.

[RNA Extraction & cDNA synthesis] Total RNA was extracted from the cells treated with “ASO 1” using RNeasy Mini kit (Qiagen, Cat. No. 714106) according to the manufacturer's instructions. cDNA was prepared from 500 ng of RNA by use of PrimeScript™ 1^{st} strand cDNA Synthesis kit (Takara, Cat. No. 6110A). To a mixture of 500 ng of RNA, 1 microliter of random hexamer, and 1 microliter of dNTP (10 mM) in PCR tube was added DEPC-treated water to a total volume of 10 microliter, which was reacted at 65° C. for 5 minutes. cDNA was synthesized by adding 10 microliter of PrimeScript RTase reaction mixture and reacting at 30° C. for 10 minutes and at 42° C. for 60 minutes, successively.

[Real-Time qPCR] In order to evaluate the expression level of human MLPH mRNA real-time qPCR was performed with synthesized cDNA by use of Taqman probe. The mixture of cDNA, Taqman probe (Thermo, Cat. No. Hs00983107_m1), IQ supermix (BioRad, Cat. No. 170-8862) and nuclease free water in PCR tube was under reaction by use of CFX96 Touch Real-Time system (BioRad) according to the cycle conditions specified as follows: at 95° C. for 3 min followed by 40 cycles of 10 sec at 95° C. and 30 sec at 60° C. . Fluorescence intensity was measured at the end of every cycle and the result of PCR was evaluated by the melting curve.

After the threshold cycle (Ct) of each gene was standardized by that of GAPDH, the relative expression level of human MLPH mRNA was compared and analyzed.

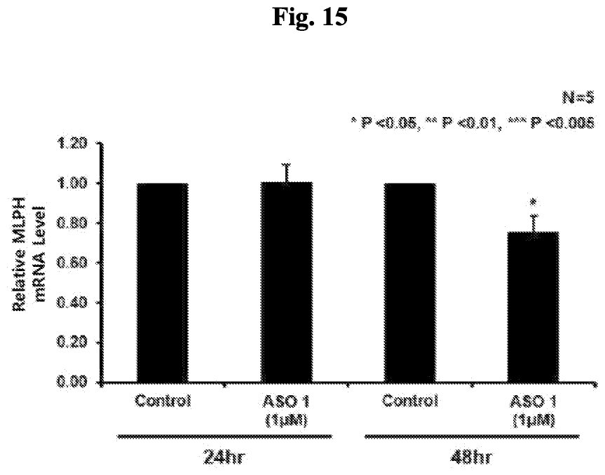

FIG. 15 provides the relative expression level of human MLPH mRNA in “ASO 1” treated cells and the level in 1μM of “ASO 1” treated cells for 48 hours was reduced. (Student T-test was done to check the statistical significance of the findings)

[Western Blotting] Cells were grown as above. After 24 and 48 hours from being treated with “ASO 1”, the cells were collected respectively, and then washed 2 times with cold PBS (phosphate buffered saline) and dissolved in RIPA buffer (Cell Signaling, Cat. No.9806). The protein was quantified with BCA solution (Thermo, Cat. No. 23225) and purified by 10% SDS-PAGE Gel. The protein was transferred on PVDF membrane (polyvinylidene fluoride membrane) (Millipore, Cat. No. IPVH00010), which was blocked in 5% skim milk buffer solution for 1 hour. The membrane was probed with an anti-MLPH (Novus, Cat. No. NBP2-45883) and anti-I3-actin (Sigma, Cat. No. A3854) as a primary antibody, and horse anti-rabbit (CST, Cat. No. 7076) was used as a secondary antibody. HRP substrate (Millipore, Cat. No. WBKLS0500) was utilized for the detection of chemiluminescent signal and the signal intensity was measured by using Gel Doc system (ATTO). Based on Western blotting results of each bands, the relative expression levels of MLPH were quantified with Image-J program and converted to the graph.

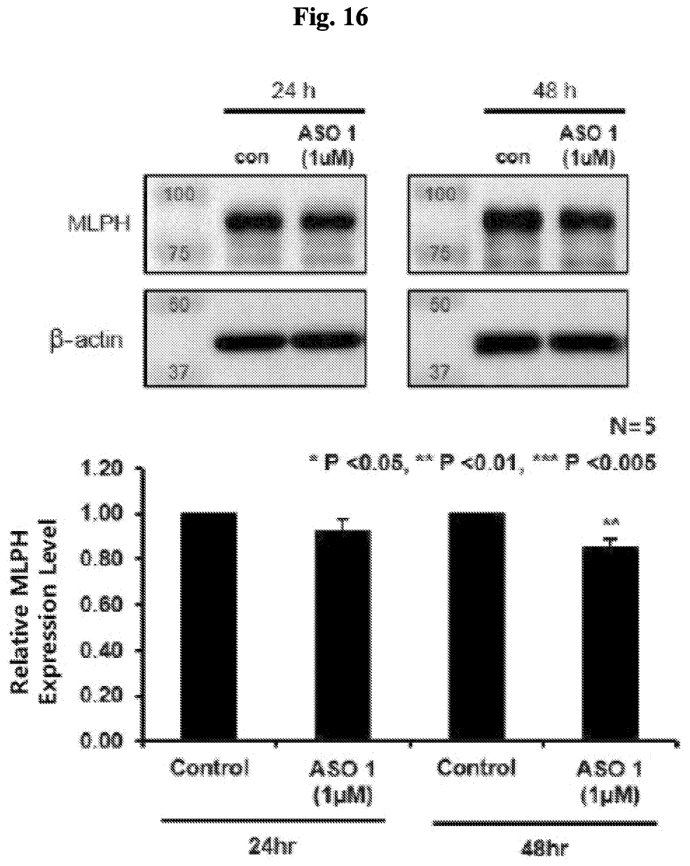

FIG. 16 provides the relative expression levels of human MLPH protein and the level in 1 μM of “ASO 1” treated cells for 48 hours was reduced. (Student T-test was done to check the statistical significance of the findings)

[Immunofluorescence Stainin g] Cells were grown as above and 48 hours after “ASO 1” treatment collected cells were fixed with 4% formaldehyde, which was blocked in 1% bovine serum albumin for an hour after permeabilization with 0.25% TritonX-100. The samples were immunostained in series with anti-tubulin (abcam, Cat. No. ab44928) and anti-Trp1 (Novus, Cat. No. NBP2-53252) as a primary antibody, and with FITC attached rabbit anti-mouse (Jackson ImmunoResearch, Cat. No.315-095-003) and Fluor 594 attached goat anti-rabbit (Jackson ImmunoResearch, Cat. No. 111-585-144) as a secondary antibody. Counter staining was performed by use of DAPI (Thermo Fisher, Cat. No. 62248) and the fluorescence digital image was evaluated with Axio Scan Z1 (Carl Zeiss) in two orders of magnitude. The ratio of the number of cells stained in red color with Trp1 near the nucleus to the number of total cells was converted to the graph.

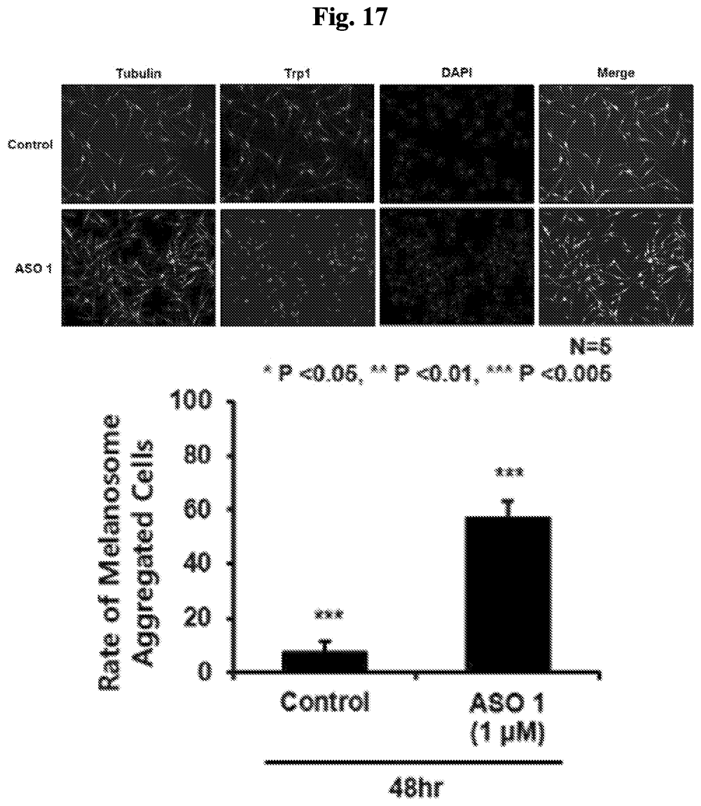

FIG. 17 provides the degree of melanosome aggregation in “ASO 1” treated cells through red color staining of Trp1. In 1 μM of “ASO 1” treated cells for 48 hours red color staining of Trp1 near DAPI stained nucleus compared to cytoskeleton tubulin suggested the melanosome aggregation by the suppression of melanosome movement. (Student T-test was done to check the statistical significance of the findings)

[Water soluble Chloro Tetrazolium-1] Cells were grown as above and water soluble chloro tetrazolium-1 assay was performed for human melanocytes (1x10³) grown in 96 well. After 24, 48, and 72 hours of 1 fM, 1 pM, 1 nM, 1 μM, and 10 μM “ASO 1” treatment, EZ-CYTOX (DoGen, Cat. No. EZ-3000) at the amount of one-tenth culture medium were added to the cells in 96 well and the resulting mixture was reacted in the incubator for 2 hours. After homogenization by shaking 1 minute at room temperature, the relative absorbance at 450 nm were measured and converted to the graph.

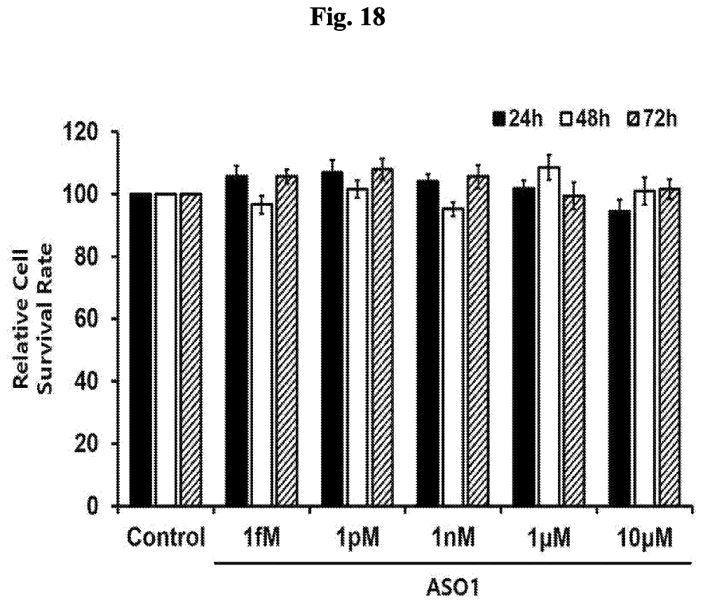

FIG. 18 provides the survival rates of “ASO 1” treated cells. After 24, 48, and 72 hours of 1 fM, 1 pM, 1 nM, 1 μM, and 10 μM “ASO 1” treatment, the survival rates of “ASO 1” treated cells were higher than 90% in every concentration after 72 hours.

1. A peptide nucleic acid derivative represented by Formula I, or a pharmaceutically acceptable salt thereof: 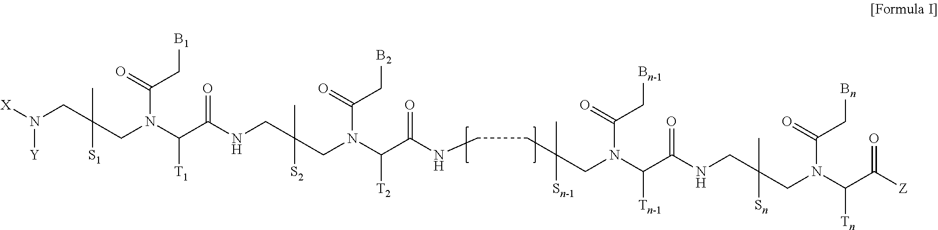
wherein,
n is an integer between 10 and 21;
the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA
the compound of Formula I is fully complementary to the human MLPH pre-mRNA, or partially complementary to the human MLPH pre-mRNA with one or two mismatches;
S₁, S₂, . . . , Sₙ₋₁, Sₙ, T₁, T₂, . . . , Tₙ₋₁, and Tₙ independently represent hydrido, deuterido, substituted or non-substituted alkyl, or substituted or non-substituted aryl radical;
X and Y independently represent hydrido, deuterido, formyl, aminocarbonyl, aminothiocarbonyl, substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;
Z represents hydrido, deuterido, hydroxy, substituted or non-substituted alkyloxy, substituted or non-substituted aryloxy, substituted or non-substituted amino, substituted or non-substituted alkyl, or substituted or non-substituted aryl radical;
B₁, B₂, . . . , Bₙ₋₁, and Bₙ are independently selected from natural nucleobases including adenine, thymine, guanine, cytosine and uracil, and unnatural nucleobases; and, at least four of B₁, B₂, . . . , Bₙ₋₁, and Bₙ are independently selected from unnatural nucleobases with a substituted or non-substituted amino radical covalently linked to the nucleobase moiety.

2. The peptide nucleic acid derivative according to claim 1, or a pharmaceutical salt thereof:
wherein,
n is an integer between 10 and 21;
the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;
the compound of Formula I is fully complementary to the human MLPH pre-mRNA, or partially complementary to the human MLPH pre-mRNA with one or two mismatches;
S₁, S₂, . . . , Sₙ₋₁, Sₙ, T₁, T₂, . . . , Tₙ₋₁, and Tₙ independently represent hydrido, deuterido radical;
X and Y independently represent hydrido, deuterido, formyl, aminocarbonyl, aminothiocarbonyl, substituted or non-substituted alkyl, substituted or non-substituted aryl, substituted or non-substituted alkyloxy, substituted or non-substituted aryloxy, substituted or non-substituted alkylacyl, substituted or non-substituted arylacyl, substituted or non-substituted alkyloxycarbonyl, substituted or non-substituted aryloxycarbonyl, substituted or non-substituted alkylaminocarbonyl, substituted or non-substituted arylaminocarbonyl, substituted or non-substituted alkylaminothiocarbonyl, substituted or non-substituted arylaminothiocarbonyl, substituted or non-substituted alkyloxythiocarbonyl, substituted or non-substituted aryloxythiocarbonyl, substituted or non-substituted alkylsulfonyl, substituted or non-substituted arylsulfonyl, substituted or non-substituted alkylphosphonyl, or substituted or non-substituted arylphosphonyl radical;
Z represents hydrido, hydroxy, substituted or non-substituted alkyloxy, substituted or non-substituted aryloxy, or substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least four of B₁, B₂, . . . , Bₙ₋₁, and Bₙ are independently selected from unnatural nucleobases represented by Formula II, Formula III, or Formula IV: 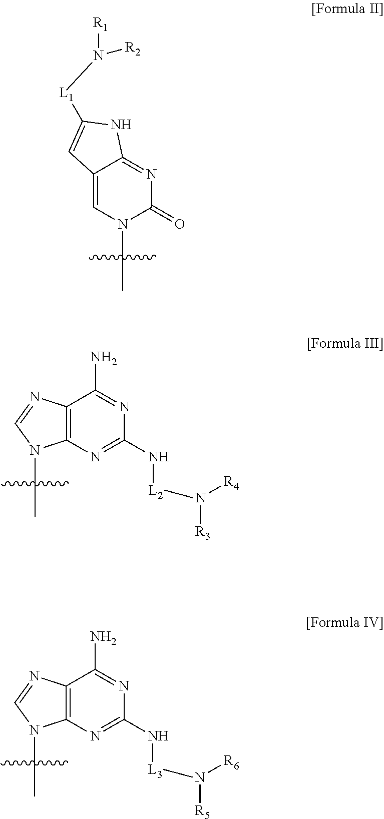
wherein,
R₁, R₂, R₃, R₄, R₅ and R₆ are independently selected from hydrido and substituted or non-substituted alkyl radical;
L₁, L₂ and L₃ are a covalent linker represented by Formula V covalently linking the basic amino group to the nucleobase moiety: 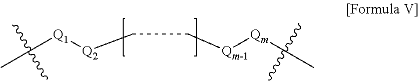
wherein,
Q₁ and Qₘ are substituted or non-substituted methylene radical [—CH₂—, —CH(substituent)-, —C(substituent)₂-], and Qₘ is directly linked to the basic amino group;
Q₂, Q₃, . . . , and Qₘ₋₁ are independently selected from substituted or non-substituted methylene, oxygen (—O—), sulfur (—S—), and substituted or non-substituted amino radical [—N(H)—, or —N(substituent)-]; and,
m is an integer between 1 and 15.

3. The peptide nucleic acid derivative according to claim 2, or a pharmaceutical salt thereof:
wherein,
n is an integer between 11 and 19;
the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;
the compound of Formula I is fully complementary to the human MLPH pre-mRNA;
S₁, S₂, . . . , Sₙ₋₁, Sₙ, T₁, T₂, . . . , Tₙ₋₁, and Tₙ are hydrido radical;
X and Y independently represent hydrido, substituted or non-substituted alkylacyl, or substituted or non-substituted alkyloxycarbonyl radical;
Z represents substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least five of B₁, B₂, . . . , Bₙ₋₁, and Bₙ are independently selected from unnatural nucleobases represented by Formula II, Formula III, or Formula IV;
R₁, R₂, R₃, R₄, R₅ and R₆ are hydrido radical;
Q₁ and Qₘ are methylene radical, and Qₘ is directly linked to the basic amino group;
Q₂, Q₃, . . . , and Qₘ₋₁ are independently selected from methylene and oxygen radical; and,
m is an integer between 1 and 9.

4. The peptide nucleic acid derivative according to claim 3, or a pharmaceutical salt thereof:
wherein,
n is an integer between 11 and 19;
the compound of Formula I possesses at least a 10-mer complementary overlap with the 30-mer pre-mRNA sequence of [(5′→3′) CCUGUGACAUUCCAGGUGUGACCCCG-ACAA] in the human MLPH pre-mRNA;
the compound of Formula I is fully complementary to the human MLPH pre-mRNA;
S₁, S₂, . . . , Sₙ₋₁, Sₙ, T₁, T₂, . . . , Tₙ₋₁, and Tₙ are hydrido radical;
X is hydrido radical;
Y represents substituted or non-substituted alkyloxycarbonyl radical;
Z represents substituted or non-substituted amino radical;
B₁, B₂, . . . , Bₙ₋₁, and Bₙ are independently selected from natural nucleobases including adenine, thymine, guanine, cytosine and uracil, and unnatural nucleobases;
at least five of B₁, B₂,. . . , Bₙ₋₁, and Bₙ are independently selected from unnatural nucleobases represented by Formula II, Formula III, or Formula IV;
R₁, R₂, R₃, R₄, R₅ and R₆ are hydrido radical;
L₁ represents —(CH₂)₂—O—(CH₂)₂—, —CH₂—O—(CH₂)₂—, —CH₂—O—(CH₂)₃—, —CH₂—O—(CH₂)₄—, or —CH₂—O—(CH₂)₅—; and,
L₂ and L₃ are independently selected from —(CH₂)₂—O—(CH₂)₂—, —(CH₂)₃—O—(CH₂)₂—, —(CH₂)₂—O—(CH₂)₃—, —(CH₂)₂—, —(CH₂)₃—, —(CH₂)₄—, —(CH₂)₅—, —(CH₂)₆—, —(CH₂)₇—, and —(CH₂)₈—.

5. The peptide nucleic acid derivative according to claim 4, which is selected from the group of peptide nucleic acid derivatives provided below, or a pharmaceutically acceptable salt thereof:
(N→C)Fethoc-GG(5)T-CA(6)C-A(6)C(1O2)C-TG(5)G-A(6)A-
NH₂;
(N→C)Fethoc-C(1O2)GG(6)-GG(6)T-CA(5)C-A(5)C(1O2)C-
TG(6)G-A(5)A-NH₂;
(N→C)Fethoc-GG(6)G-G(6)TC-A(5)CA(5)-C(1O2)CT-
G(6)GA(5)-ATG(6)-NH₂;
and
(N→C)Fethoc-GG(6)T-CA(5)C-A(5)C(1O2)C-TG(6)G-
A(5)AT-G(6)TC(1O2)-NH₂
wherein,
A, T, G and C are monomers of peptide nucleic acid with a natural nucleobase of adenine, thymine, guanine and cytosine, respectively;
C(pOq), A(p), and G(p) are monomers of peptide nucleic acid with an unnatural nucleobase represented by Formula VI, Formula VII, and Formula VIII, respectively; 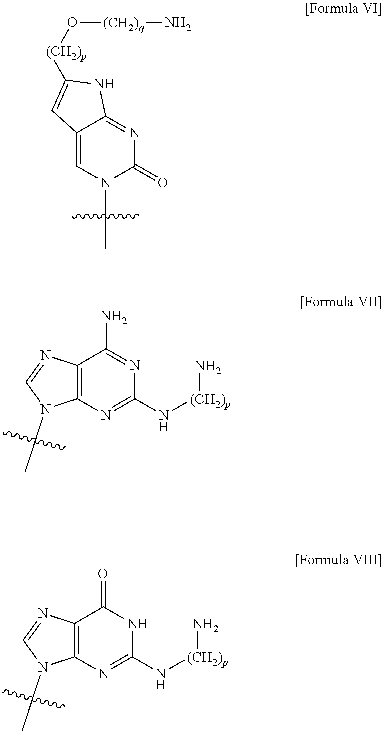
wherein,
p and q are integers, p is 1, 5, or 6 and q is 2 in (N→C)
Fethoc-GG(5)T-CA(6)C-A(6)C(1O2)C-TG(5)G-A(6)A-NH₂; and,
“Fethoc-” is the abbreviation for “[2-(9-fluorenyl)ethyl-1-oxy]carbonyl”.

6. A method to treat diseases or conditions associated with the human MLPH gene transcription, comprising the administration of the peptide nucleic acid derivative according to claim 1, or a pharmaceutically acceptable salt thereof to a subject.

7. A method to treat skin pigmentation, comprising the administration of the peptide nucleic acid derivative according to claim 1, or a pharmaceutically acceptable salt thereof to a subject.

8. A pharmaceutical composition for treating diseases or conditions associated with human MLPH gene transcription, comprising the peptide nucleic acid derivative according to claim 1, or a pharmaceutically acceptable salt thereof.

9. A cosmetic composition for treating diseases or conditions associated with human MLPH gene transcription, comprising the peptide nucleic acid derivative according to claim 1, or a pharmaceutically acceptable salt thereof.

10. A pharmaceutical composition for treating skin pigmentation, comprising the peptide nucleic acid derivative according to claim 1, or a pharmaceutically acceptable salt thereof.

11. A cosmetic composition for treating skin pigmentation, comprising the peptide nucleic acid derivative according to claim 1, or a pharmaceutically acceptable salt thereof.


SEQUENCE LISTING (14 sequences)

SEQ ID NO: 1 - DNA, 14 bp, Artificial
  PNA modified oligonucleotide
  misc_feature (1)..(14): PNA modified oligonucleotide, human melanophilin targeted artificial sequence
  misc_feature (1)..(1): Carbamated N-terminal
  modified_base (2)..(2): n is a, g, c, or t
  modified_base (5)..(5): n is a, g, c, or t
  modified_base (7)..(7): n is a, g, c, or t
  modified_base (8)..(8): n is a, g, c, or t
  modified_base (11)..(11): n is a, g, c, or t
  modified_base (13)..(13): n is a, g, c, or t
gntcncnnctngna

SEQ ID NO: 2 - DNA, 17 bp, Artificial
  PNA modified oligonucleotide
  misc_feature (1)..(17): PNA modified oligonucleotide, human melanophilin targeted artificial sequence
  misc_feature (1)..(1): Carbamated N-terminal
  modified_base (1)..(1): n is a, g, c, or t
  modified_base (3)..(3): n is a, g, c, or t
  modified_base (5)..(5): n is a, g, c, or t
  modified_base (8)..(8): n is a, g, c, or t
  modified_base (10)..(10): n is a, g, c, or t
  modified_base (11)..(11): n is a, g, c, or t
  modified_base (14)..(14): n is a, g, c, or t
  modified_base (16)..(16): n is a, g, c, or t
ngngntcncnnctngna

SEQ ID NO: 3 - DNA, 18 bp, Artificial
  PNA modified oligonucleotide
  misc_feature (1)..(18): PNA modified oligonucleotide, human melanophilin targeted artificial sequence
  misc_feature (1)..(1): Carbamated N-terminal
  modified_base (2)..(2): n is a, g, c, or t
  modified_base (4)..(4): n is a, g, c, or t
  modified_base (7)..(7): n is a, g, c, or t
  modified_base (9)..(9): n is a, g, c, or t
  modified_base (10)..(10): n is a, g, c, or t
  modified_base (13)..(13): n is a, g, c, or t
  modified_base (15)..(15): n is a, g, c, or t
  modified_base (18)..(18): n is a, g, c, or t
gngntcncnnctngnatn

SEQ ID NO: 4 - DNA, 18 bp, Artificial
  PNA modified oligonucleotide
  misc_feature (1)..(18): PNA modified oligonucleotide, human melanophilin targeted artificial sequence
  misc_feature (1)..(1): Carbamated N-terminal
  modified_base (2)..(2): n is a, g, c, or t
  modified_base (5)..(5): n is a, g, c, or t
  modified_base (7)..(7): n is a, g, c, or t
  modified_base (8)..(8): n is a, g, c, or t
  modified_base (11)..(11): n is a, g, c, or t
  modified_base (13)..(13): n is a, g, c, or t
  modified_base (16)..(16): n is a, g, c, or t
  modified_base (18)..(18): n is a, g, c, or t
gntcncnnctngnatntn

SEQ ID NO: 5 - DNA, 14 bp, Artificial
  PNA modified oligonucleotide
  misc_feature (1)..(14): PNA modified oligonucleotide, mouse melanophilin targeted artificial sequence
  misc_feature (1)..(1): Carbamated N-terminal
  modified_base (2)..(2): n is a, g, c, or t
  modified_base (5)..(5): n is a, g, c, or t
  modified_base (7)..(7): n is a, g, c, or t
  modified_base (8)..(8): n is a, g, c, or t
  modified_base (11)..(11): n is a, g, c, or t
  modified_base (13)..(13): n is a, g, c, or t
gnccncnnctngna

SEQ ID NO: 6 - DNA, 17 bp, Artificial
  PNA modified oligonucleotide
  misc_feature (1)..(17): PNA modified oligonucleotide, mouse melanophilin targeted artificial sequence
  misc_feature (1)..(1): Carbamated N-terminal
  modified_base (1)..(1): n is a, g, c, or t
  modified_base (3)..(3): n is a, g, c, or t
  modified_base (5)..(5): n is a, g, c, or t
  modified_base (8)..(8): n is a, g, c, or t
  modified_base (10)..(10): n is a, g, c, or t
  modified_base (11)..(11): n is a, g, c, or t
  modified_base (14)..(14): n is a, g, c, or t
  modified_base (16)..(16): n is a, g, c, or t
ncngnccncnnctngna

SEQ ID NO: 7 - DNA, 18 bp, Artificial
  PNA modified oligonucleotide
  misc_feature (1)..(18): PNA modified oligonucleotide, mouse melanophilin targeted artificial sequence
  misc_feature (1)..(1): Carbamated N-terminal
  modified_base (2)..(2): n is a, g, c, or t
  modified_base (5)..(5): n is a, g, c, or t
  modified_base (7)..(7): n is a, g, c, or t
  modified_base (8)..(8): n is a, g, c, or t
  modified_base (11)..(11): n is a, g, c, or t
  modified_base (13)..(13): n is a, g, c, or t
  modified_base (15)..(15): n is a, g, c, or t
  modified_base (18)..(18): n is a, g, c, or t
gnccncnnctngnangtn

SEQ ID NO: 8 - DNA, 18 bp, Artificial
  PNA modified oligonucleotide
  misc_feature (1)..(18): PNA modified oligonucleotide, mouse melanophilin targeted artificial sequence
  misc_feature (1)..(1): Carbamated N-terminal
  modified_base (2)..(2): n is a, g, c, or t
  modified_base (4)..(4): n is a, g, c, or t
  modified_base (7)..(7): n is a, g, c, or t
  modified_base (9)..(9): n is a, g, c, or t
  modified_base (10)..(10): n is a, g, c, or t
  modified_base (13)..(13): n is a, g, c, or t
  modified_base (15)..(15): n is a, g, c, or t
  modified_base (17)..(17): n is a, g, c, or t
cngnccncnnctngnang

SEQ ID NO: 9 - RNA, 30 nt, Homo sapiens
ccugugacauuccaggugugaccccgacaa

SEQ ID NO: 10 - RNA, 30 nt, Mus musculus
ccugugacuuucuagguguggccuggauga

SEQ ID NO: 11 - DNA, 20 bp, Artificial
  Synthetic primer
tagctcagtgcaccctgaca

SEQ ID NO: 12 - DNA, 20 bp, Artificial
  Synthetic primer
gagagaccggatcactttgg

SEQ ID NO: 13 - RNA, 19 nt, Artificial
  Mouse MLPH targeted siRNA
gggcaaaauacaaaaggag

SEQ ID NO: 14 - RNA, 19 nt, Artificial
  Mouse MLPH targeted siRNA
cuccuuuuguauuuugccc